(19) United States
(12) Patent Application Publication
(10) Pub. No.: US 2025/0282839 A1
(43) Pub. Date: Sep. 11, 2025

(54) PURIFIED MUCUS

(71) Applicant: Aelius Biotech Limited, Newcastle upon Tyne (GB)

(72) Inventors: Peter Chater, Newcastle Upon Tyne (GB); Matthew Wilcox, Newcastle Upon Tyne (GB); Kyle Stanforth, Newcastle Upon Tyne (GB); Maria Zakhour, Newcastle Upon Tyne (GB); Jeffrey Pearson, Newcastle Upon Tyne (GB)

(73) Assignee: Aelius Biotech Limited, Newcastle upon Tyne (GB)

(21) Appl. No.: 18/861,190

(22) PCT Filed: Apr. 28, 2023
(86) PCT No.: PCT/GB2023/051145

§ 371 Date: Oct. 28, 2024

(30) Foreign Application Priority Data
Apr. 29, 2022 (GB) 2206352.3

(51) Int. Cl. C07K 14/47; C12N 5/071; G01N 33/02; G01N 33/50

(52) U.S. Cl. CPC C07K 14/4727; C12N 5/0679; C12N 5/0697; G01N 33/02; G01N 33/5005; C12N 2503/04; C12N 2533/50

(57) ABSTRACT

The present invention relates to a purified, biocompatible mucus, which has the ability to form a gel. The mucus of the invention may be provided in a tissue model, with a cell population. The mucin or tissue model of the invention may be useful in an in vitro model of digestion, mucus permeation and/or epithelial transport.

This invention relates to a purified, biocompatible mucus, and uses thereof. The present invention also relates to a tissue model comprising a purified, biocompatible mucus and a cell; and uses thereof. The present invention also relates to a method of purifying mucus.

### BACKGROUND

For successful oral uptake of an active pharmaceutical agent, the compound must pass through a series of barriers and retain sufficient yield to have therapeutic effect. Firstly the compound must overcome the enzymatic, chemical, and physical challenges during the digestive phases in the mouth, stomach, and small intestine, including for example digestive-enzymes, pH changes, and components such as bile which may interact and impede the pharmaceutical agent. Even once it reaches the small intestine, the drug delivery system must then permeate the mucus barrier for the agent to successfully reach the epithelia where it is taken up into the blood stream. The mucus layer functions as a barrier, excluding or trapping certain compounds on the basis of size and charge. Any such particles are removed as the mucus layer is turned over. As a result, oral delivery of drugs, including peptide and protein therapeutics, can be impeded by the presence of the mucus surface-lining the intestinal epithelium. The mucus is made up of 95% water and a gel forming glycoprotein, mucin, and in some mucus, lipid, DNA and actin may be present.

It is important to test the oral delivery of drugs in vitro before in vivo testing. This may provide an alternative to reduce, refine or replace the need for animal models. For this reason, individual in vitro models exist for different phases of the digestive system.

WO 2015075467 and Houghton et al (Food Chem. 2014 May 15; 151:352-7) describe a simulated model of the digestive tract cumulative of the mouth stomach and small intestine, using physiologically relevant secretions and whole pig bile collected from an abbatoir to provide a model closely pertaining to the human digestive tract. Friedl et al (J Pharm Sci. 2013 December; 102 (12): 4406-13) describe an in vitro transwell mucus permeation model used to simulate nanoparticle diffusion through the mucus membrane. In addition, a number of cell culture systems are available to model the intestinal epithelia. Until now it has been impossible to look at the whole process together, and how these phases interact with each other.

WO2018/175861 describes a macro tissue explant for use in high throughput screening, which comprises a mammalian tissue explant in a multi-well substrate. The tissue explant may additionally comprise a mucus layer.

Gleeson & McCartney (Trends in pharmacological sciences 40.10 (2019): 720-724) describes the co-culturing of Caco-2 monolayers with HT29-MTX-E12, where HT29 cells are another intestinal cell line that is differentiated into mature goblet cells using methotrexate. The H29 cell line was developed to mimic the mucus-lined epithelium. However, this is not an optimum solution because the mucus layer is 4 μm, which is significantly thinner than the mucus layers in the thinnest region of the human intestine (typically around 15 μm). This means that even co-culture systems cannot be integrated with whole digestive fluids due to the likelihood of cell death.

It has been known to use porcine mucus in such in vitro systems. However, although porcine mucus reflects human intestinal mucus, Caco-2 cells do not survive prolonged co-incubation. To overcome this problem, a biosimilar mucus has been developed. Biosimilar mucus, which is compatible with Caco-2 monolayers, mimics the rheological properties of porcine intestinal mucus and has been used to assess drug absorption in vitro. Boegh, Marie, et al. (European Journal of Pharmaceutics and Biopharmaceutics 87.2 (2014): 227-235) describe the design and characterization of biosimilar mucus compatible with Caco-2 cell monolayers cultured in vitro to establish a more representative in vitro model for the intestinal mucosa. The biosimilar mucus mixture is composed of degraded commercial gastric mucin, BSA, cholesterol, phosphatidylcholine, linoleic acid, and polyacrylic acid (Carbopol 974P NF at 0.3 to 0.9% w/v). Polyacrylic acid is a synthetic polymer, which is added to the composition to provide the desired viscoelastic properties and a microstructure comparable to freshly isolated porcine intestinal mucus (PIM). The biosimilar mucus was optimized with regard to the lipid content in order to obtain cellular compatibility with well-differentiated Caco-2 cell monolayers. However, significant differences in peptide permeability were found between the isolated PIM and the biosimilar mucus. In addition, Carbopol is known to interact with mucin, and so will alter mucus properties and/or function. Commercially available porcine gastric mucin (Sigma Aldrich™) has been denatured during isolation, and consequently its ability to form mucin: mucin interactions is lost, which results in its inability to form a gel. Furthermore, gastric mucin is comprised of MUC5AC and MUC6 gene products, whereas small intestinal mucin is MUC2.

There are a number of problems with other approaches to modelling intestinal tissue comprising a mucus layer. Human mucus is difficult to obtain, and furthermore the isolation process can alter the mucins. In addition, synthetic gastro-intestinal secretions are used to mimic the native intestinal environment, but such synthetic secretions have been found to cause cell death when applied to cell cultures in the absence of a protective mucus layer as would be found in vivo. Native porcine mucus collected from animals has also been found to kill cells, and therefore is not suitable for use in a tissue model.

The present invention aims to overcome or ameliorate some or all of these problems associated with the prior art.

### BRIEF SUMMARY OF THE DISCLOSURE

The present invention provides, in a first aspect, a purified, biocompatible mucus, wherein the mucus:
i) substantially lacks nucleic acids, protein and/or lipids, and/or molecules below 100 kDa, preferably below 70 kDa; and ii) comprises mucin, wherein the mucin has the ability to form a gel. Suitably, the purified, biocompatible mucus contains mucin which has a density of 1.3 to 1.6 g/mL. Suitably, the purified, biocompatible mucus does not comprise a synthetic polymer. Suitably, the purified, biocompatible mucus contains mucin, and wherein the mucin is not substantially denatured, hydrolysed or degraded. Suitably, the mucin present in the mucus comprises sufficient intermolecular interactions to enable the purified biocompatible mucus to form a gel. In an embodiment, the mucus is a gel.

The present invention also provides, in a second aspect, a tissue model, comprising:
a substrate having a cell support portion;
a cell population provided on the substrate;
a purified, biocompatible mucus according to the first aspect;
optionally, a fluid provided on the mucus layer and/or below the substrate layer.

In a third aspect, there is provided a method of purifying mucus, wherein the method comprises removal of substantially all nucleic acids, protein and/or lipids, and/or molecules below 100 kDa, preferably below 70 kDa from the mucus, wherein the step of removal comprises dialysis and/or equilibrium density gradient centrifugation; to provide a purified, biocompatible mucus. Suitably, the method does not substantially remove mucin from the mucus. Suitably, the method does not comprise denaturation, degradation or hydrolysis of mucin. Optionally, the method may comprise a further separation step, for example centrifugation. Suitably, the method provides a purified biocompatible mucus of the first aspect.

In an embodiment of the third aspect, the method may comprise i) providing a sample comprising mucus; ii) solubilising any mucin in the mucus; iii) separating from the mucus any nucleic acids, proteins, and/or lipids; and optionally iv) removing any contaminants, undesirable components or reagents from the mucus; to provide a purified, biocompatible mucus. Step iii) may comprise equilibrium density gradient centrifugation. Step iv) may comprise removing any cytotoxic components, reagents, and/or enzymes, and may comprise dialysis of the mucus. Suitably, the method comprises separating from the mucus a fraction having a density gradient of 1.3 to 1.6 g/mL. Suitably, the mucin present in the mucus has a density of 1.3 to 1.6 g/mL.

In an embodiment of the third aspect, the method may comprise i) providing a sample comprising mucus; ii) dialysing the mucus to separate from the mucus any molecules below 100 kDa, preferably below 70 kDa; and optionally iii) separating the mucus.

In a fourth aspect, there is provided a method for determining or predicting absorption of a test compound through a tissue model, wherein the tissue model comprises i) a substrate comprising a cell support portion; ii) a cell population in contact with the cell support portion of the substrate; and iii) purified biocompatible mucus, in contact with the cell; and wherein the method comprises contacting the purified biocompatible mucus with the test compound, and detecting movement of the test compound in the tissue model; thereby determining the ability of the test compound to traverse the purified biocompatible mucus and cellular layers of the tissue model.

In an embodiment of the fourth aspect, the tissue model may comprise purified biocompatible mucus or a composition comprising the purified biocompatible mucus as described in the first aspect of the invention. The tissue model may be as described in the second aspect of the invention. The tissue model used in a method of detecting an effect of a test compound may be a stomach tissue model, small intestine tissue model, large intestine tissue model, gastro-intestinal tissue model, female reproductive tissue model, nasal tissue model, optic tissue model and/or airway tissue model.

The step of detecting movement of the test compound in the tissue model may comprise observing, monitoring or measuring any one or more of, but not limited to, the distance travelled by the test compound into the tissue model, the absorption of the test compound into the tissue model, the path travelled in the tissue model, the rate of travel or perfusion into the tissue model, cellular uptake in the tissue model, and the location of the test compound in the tissue model. The detecting may also comprise determining the concentration of the test compound at or below the substrate. The step of detecting the movement of the test compound may comprise detecting the test compound in the purified biocompatible mucus layer, the cell population, and/or in contact with a surface of the substrate. The presence or absence of the test compound at the luminal surface and/or at the basolateral surface of the tissue mode may be detected. The presence of the test compound at the basolateral surface indicates the ability of the compound to be absorbed through the tissue explant.

The method may comprise the step of applying food to the purified biocompatible mucus or fluid layer of the model. The method may comprise the step of applying native intestinal media to the purified biocompatible mucus or fluid layer of the model.

In a fifth aspect, there is provided a kit comprising, in a suitable container, purified biocompatible mucus or a composition comprising the purified biocompatible mucus as described in the first aspect, and/or a tissue model of the second aspect, and optionally a substrate, a buffer, reagents, instructions for use, and other standard ingredients well known in the art.

### BRIEF DESCRIPTION OF THE DRAWINGS

Embodiments of the invention are further described hereinafter with reference to the accompanying drawings, in which:

FIG. 1 is a graph showing Caco-2 cell viability in the presence of simulated digestive fluids with and without native mucus in a 24 well plate transwell over a period of 4 hours. The Cytotoxic effects of porcine small intestinal surface mucus on a Caco-2 cell monolayer, with either luminal PBS or simulated small intestinal fluids are shown with a Cell titre Blue cell viability assay. Native mucus kills cells, but confers some protection of the remaining live cells by GI fluids. Under agitation, the ability of native mucus to protect cells is diminished.

FIG. 2 is a representation of the problems with viscosity of commercial mucins. Human mucus is difficult to obtain. The commercial isolation process alters the mucin glycoproteins and disrupts the ability to form mucin: mucin interactions. Purification methods including boiling, or exposure to proteolytic or glycolytic enzymes can alter mucins. The purification method described herein in the third aspect does not affect mucin: mucin interactions, and allows mucin to retain the ability to form a gel at higher concentrations.

FIG. 3 is a diagrammatic representation of an example of how a model may be set up according to the present invention.

FIG. 4 shows a Pearson's correlation of 17 Active Pharmaceutical Ingredients (API's) permeated through both 10 uL native mucus and 50 μL of 75 mg/mL reconstituted small intestinal mucin. A strong positive correlation (R²=0.8517) was observed.

FIG. 5 shows the rheological properties of the purified mucus of the invention, rehydrated in phosphate buffered saline at concentrations of 5-200 mg/ml, compared to native small intestine porcine mucus and sodium chloride cleaned porcine small intestinal mucus. Panel A and B show the maximum shear stain and stress respectively in the Linear viscoelastic region. Panel C, D and E show the storage modulus, loss modulus and phase angle within the LVER. Panel F shows the shear strain required to breakdown the down the gel to a viscous liquid (yield point) and allow it to flow. Experiments were repeated a minimum of three times with separate batches of mucus and mucin.

FIG. 6 shows the effect of native porcine mucus on cell viability of Caco-2 cells grown in 96 well, dual chamber model as per FIG. 3. Native porcine mucus significantly reduces cell viability in 96 well plate models.

FIG. 7 shows the effect of concentration and volume of the purified biocompatible mucus, reconstituted in buffer (referred to herein as reconstituted mucus or RM), on compatibility with Caco-2 cells. In the same model as used in FIG. 6, a range of concentrations and volumes of reconstituted mucus were tested for their compatibility with caco2 cells with applied simulated small intestinal secretions for a length of 4 hours. 50 and 70 μL of all concentrations were not significantly different from the control, indicating that these protect cells from the simulated digestive secretions in vitro over a time course of 4 hours.

FIG. 8 shows the effect of the reconstituted mucus of the invention in protecting against the effects of simulated small intestinal digestive fluids. In a 24 well plate model, 50 μL of 25, 50 and 75 mg/ml was shown to protect cells from simulated small intestinal digestive fluids over a 4-hour time course.

FIG. 9 shows levels of cell viability of gastric cell line CRL-1739 integrated with a biocompatible mucus layer and gastric secretions. Gastric epithelial cells were overlayered with gastric mucus layers of varying thicknesses and mucin concentrations, and exposed to model gastric digestive secretions.

FIG. 10 shows the impact on airway cell viability of the purified mucus of the invention at various volumes and concentrations, for 90 minutes. Panel A shows cell viability groups with volume of liquid added to the apical surface of Calu3 airway cells with concentration of mucin show with the bars. Panel B shows the same data but groups with concentration of mucin applied with the bars showing varying volume. All experiments were repeated a minimum of four times.

FIG. 11 shows that cleaned porcine small intestinal mucus dialysed in 50,000 Da MWCO tubing is a cell compatible gel, and protects the cells from simulated small intestinal fluids for 2 hours. The same result is achieved when mucus is spun for 1.5 hours after dialysis to remove excess water.

FIG. 12 shows the effect of various sterilisation solvents on 5 mg/ml gastric mucus after incubation with gastric cells for 24 hours

FIG. 13 shows the colony forming units present in the mucus after sterilisation with various solvents

FIG. 14 Caco-2 cell viability of cells incubated with a layer of RM at various concentrations and volumes, with EPDF applied to the surface of the mucus layer (n=3). Cells were incubated with RM and EPDF for 1 hour (top left), 2 hours (top right), 3 hours (bottom left) and 4 hours (bottom right). Included in the analysis was a live control, which had been exposed to PBS, and a 0 mucus control (i.e a model without mucus, but with the fluids, e.g digestive fluids, present), which was exposed to EPDF. The y-axis shows the cell viability in % determined via a Cell TiterBLue assay. The y-axis shows the live control and the respective volumes of RM. The black bar shows the live control, the dark grey bar represents 25 mg/mL RM, the light grey bar represents 50 mg/mL RM, and the white bar represents 75 mg/mL RM. Bars indicated with an asterisk are significantly different to the live control. For the 2 hours graph, bars that show a pair of asterisks indicate groups which share a significance group with the no mucus control and the live control.

FIG. 15 Graph depicting all apparent permeability values for all APIs for all mucus concentrations and volumes. The y-axis shows the apparent permeability of APIs, expressed in cm/s×10⁻⁶. The x-axis shows the API. The x-axis also has two legends, one which indicates the Log D or Log P value of an API, and another which indicates an APIs charge. APIs are grouped based on their physiological charge and permeability values. A legend is shown within the figure which indicates which mucus condition is applicable to each bar. PNM stands for processed native mucus, which is cleaned porcine small intestinal mucus. RM X (X) represents reconstituted mucus volumes (outside bracket) in uL and concentrations (inside bracket) in mg/mL. APIs with higher Log D or Log P values and positive charges have apparent permeability values typically far lower than those which do not.

FIG. 16 Pearson's correlation of the apparent permeability of 11 APIs through 20, 50 and 70 μL of 25 mg/mL reconstituted mucus against 10, 15 and 20 μL of PNM (Processed/Cleaned Native Mucus). Permeations were repeated three times. The y-axis shows the apparent permeability of APIs through the RM. The x-axis shows the apparent permeability of APIs through PNM. Apparent permeability is expressed as cm·s⁻¹×10⁻⁶. (A) 20 μL RM vs 10 μL PNM. (B) 50 μL RM vs 10 μL PNM. (C) 70 μL RM vs 10 μL PNM (D) 20 μL RM vs 15 μL PNM. (E) 50 μL RM vs 15 μL. (F) 70 μL RM vs 15 μL. (G) 20 μL RM vs 20 μL PNM. (H) 50 μL RM vs 20 μL PNM. (I) 70 μL RM vs 20 μL PNM. All relationships

FIG. 17 Correlation of the apparent permeability of 11 APIs through 20, 50 and 70 μL of 75 mg/mL reconstituted mucus against 10, 15 and 20 μL of PNM (Processed/Cleaned Native Mucus). The y-axis shows the Pₐₚₚ of APIs through RM. The x-axis shows the apparent permeability of APIs through PNM. Apparent permeability is expressed as cm·s⁻¹×10⁻⁶. (A) 20 μL RM vs 10 μL PNM. (B) 50 μL RM vs 10 μL PNM. (C) 70 μL RM vs 10 μL PNM (D) 20 μL RM vs 15 μL PNM. (E) 50 μL RM vs 15 μL. (F) 70 μL RM vs 15 μL. (G) 20 μL RM vs 20 μL PNM. (H) 50 μL RM vs 20 μL PNM. (I) 70 μL RM vs 20 UL PNM. Figures C, F and I were Spearman's Rank correlations, the others were Pearson's. All relationships bar that of FIG. 1 were

FIG. 18 Pearson's correlation of the apparent permeability of 11 APIs through 20, 50 and 70 μL of 50 mg/mL reconstituted mucus against 10, 15 and 20 μL of PNM (Processed/Cleaned Native Mucus). Permeations were repeated three times. The y-axis shows the apparent permeability of APIs through RM. The x-axis shows the apparent permeability of APIs through PNM. Apparent permeability is expressed as cm·s¹×10⁻⁶. (A) 20 μL RM vs 10 μL PNM. (B) 50 μL RM vs 10 μL PNM. (C) 70 μL RM vs 10 μL PNM (D) 20 μL RM vs 15 μL PNM. (E) 50 μL RM vs 15 μL. (F) 70 μL RM vs 15 μL. (G) 20 UL RM vs 20 μL PNM. (H) 50 μL RM vs 20 μL PNM. (I) 70 μL RM vs 20 μL PNM. All relationships were significant.

FIG. 19 Pearson's correlation of the API apparent permeability data set with positively charged APIs removed from the data set. The y-axis shows API apparent permeability through reconstituted. The x-axis shows apparent permeability through PNM (Processed/Cleaned Native Mucus). Apparent permeability is expressed in cm/s×10-6. A legend is shown above each column of figures (for PNM volume) and within each figure (for RM concentration and volume). All relationships were significant (p<0.05). R2 values indicate how well the data fits into the correlation. Correlations ranged in strength, with the best relationships observed at 50 μL of 50 mg/ml against 10 and 15 μL PNM (0.87 and 0.80 respectively), and 50 μL of 75 mg/mL against 10 μL PNM (0.83).

FIG. 20 Pearson's correlation of the API apparent permeability data set with APIs with Log P>3 removed from the data set. The y-axis shows API apparent permeability through reconstituted mucus. The x-axis shows apparent permeability through PNM (Processed/Cleaned Native Mucus). Apparent permeability is expressed in cm/s×10⁻⁶. A legend is shown above each column of figures (for PNM volume) and within each figure (for RM concentration and volume). All relationships were significant (p<0.05). R² values indicate how well the data fits into the correlation. Correlations ranged in strength, with the best relationships observed at 10 μL of PNM (50 μL of 50 mg/mL, R²=0.92; 70 μL of 50 mg/mL, R²=0.87; 50 μL of 75 mg/mL, R²=0.88).

FIG. 21 Spearman's Rank correlation analysis of Caco-2 cell Pₐₚₚ of 13 APIs permeated through the integrated model, and Human Jejunal P_{eff}. The y-axis shows the measured Caco-2 cell Pₐₚₚ, measured in cm/s×10⁻⁶. The y-axis shows the Human jejunum P_{eff} value expressed in cm/s×10⁻⁶. Integrated model runs were carried using reconstituted mucus concentrations and volumes of either 50 μL of 25 mg/mL, 50 or 70 μL of 50 mg/mL, or 50 μL of 75 mg/mL. Interpretation of R² is as follows: medium strength has an R² value of between 0.4 and 0.69, and a high strength correlation has an R² value of between 0.7 and 0.89. In all correlations a significant relationship was observed (p<0.05). (A) 50 μL of 25 mg/mL RM vs P_{eff}. A medium strength correlation was observed, indicated by the R² value of 0.6480. The equation of the trendline was y=0.0011x+0.1229. (B) 50 μL of 50 mg/mL RM vs P_{eff}. A medium strength correlation was observed, indicated by the R² value of 0.6839. The equation of the trendline was y=0.0014x+0.3123. (C) 70 μL of 50 mg/mL RM vs P_{eff}. A medium strength correlation was observed, indicated by the R² value of 0.68. The equation of the trendline was y=0.0011x+0.1229. (D) 50 μL of 75 mg/mL RM vs P_{eff}. A strong correlation was observed, indicated by the R² value of 0.70. The equation of the trendline was y=0.0012x+0.1957.

FIG. 22 Spearman's Rank correlation analysis of Caco-2 cell Pₐₚₚ of 8 passively absorbed APIs permeated through the integrated model, and Human Jejunal P_{eff}. The y-axis shows Caco-2 cell Pₐₚₚ in cm/s×10⁻⁶. The y-axis shows the Human jejunal P_{eff} value expressed in cm/s×10⁻⁶. Integrated model runs were carried using RM concentrations and volumes of either 50 μL of 25 mg/mL, 50 or 70 μL of 50 mg/mL, or 50 μL of 75 mg/mL. Interpretation of R² values can be observed in Section 2.2.19. Interpretation of R² is as follows: medium strength has an R² value of between 0.4 and 0.69, and a high strength correlation has an R² value of between 0.7 and 0.89. In all correlations a significant relationship was observed (p<0.05). (A) 50 μL of 25 mg/mL RM vs P_{eff}. A high strength correlation was observed, indicated by the R² value of 0.86. The equation of the trendline was y=0.0038x+0.64. (B) 50 μL of 50 mg/mL RM vs P_{eff}. A medium correlation was observed, indicated by the R² value of 0.65. The equation of the trendline was y=0.0015x+0.3187. (C) 70 μL of 50 mg/mL RM vs P_{eff}. A medium correlation was observed, indicated by the R² value of 0.6590. The equation of the trendline was y=0.0012x+0.1426. (D) 50 μL of 75 mg/mL RM vs P_{eff}. A high strength correlation was observed, indicated by the R² value of 0.71. The equation of the trendline was y=0.0012x+0.1943.

FIG. 23 Caco-2 cell viability of cells incubated with DM (300), either neat or diluted 1:1 prior to dialysis, with PBS or EDPF for 1, 2, 3 or 4 hours. The x-axis shows the mucus group. The y-axis shows the Caco-2 cell viability, expressed in %. The legend within the figure indicates the time point of the analysis. Statistics bars indicate where mean viability values were statistically different from one another. Statistics was determined with a two-way ANOVA test. The asterisk indicates the significance level of the data. A single asterisk shows where p<0.05. ANOVA testing was carried out only at individual time points. Comparison of groups between time points is not valid.

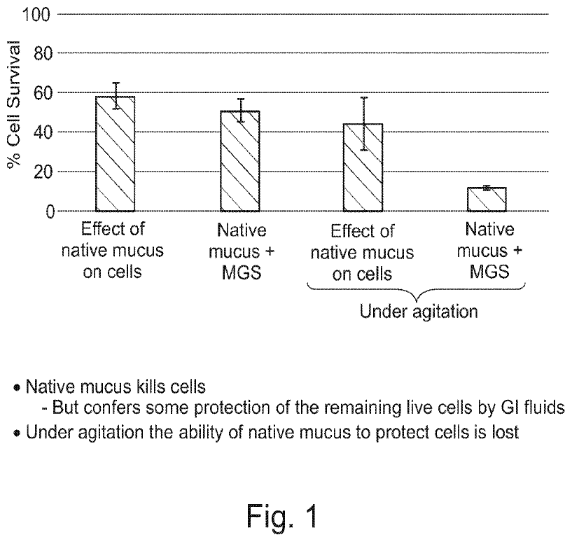

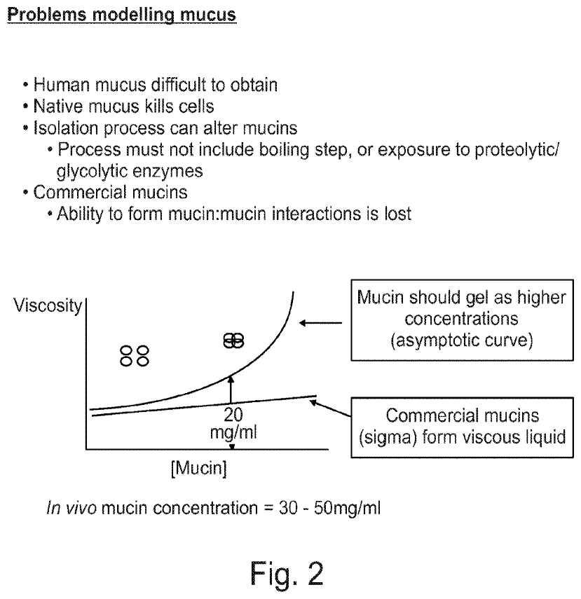

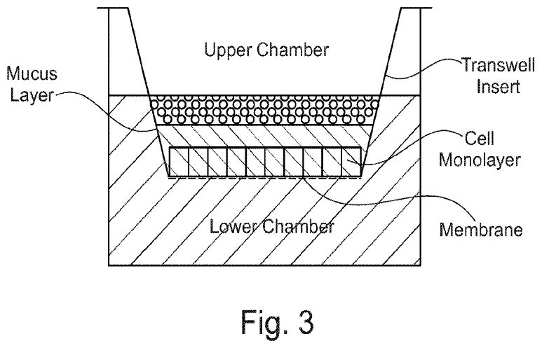

### DETAILED DESCRIPTION

The present invention is based on the surprising finding by the inventors that it is possible to use dialysis and/or equilibrium density gradient centrifugation to obtain a purified, biocompatible mucus, which retains the ability to form a gel. The method of the invention does not degrade, hydrolyse or denature the mucin of the mucus, and therefore allows the mucin to retain the ability to form mucin-mucin interactions, and therefore provide viscoelastic properties to the purified mucus. The purified mucus is therefore able to form a gel. The purified mucus has also been shown by the present inventors to be biocompatible (also referred to as biologically compatible), such that it can be applied to cells without having a cytotoxic effect. Therefore the present invention provides for the first time a mucin preparation which is suitable for use in an in vitro model of digestion, mucus permeation and/or epithelial transport.

### Definitions

It must be noted that, as used in the specification and the claims, the singular forms “a,” “an,” and “the” include plural referents unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The terms “comprise”, “comprises” and “comprising” as used herein, specify the presence of the stated features, integers, steps, operations, elements, and/or components of an aspect or embodiment of the invention, but do not preclude the presence or addition of one or more other features, integers, steps, operations, elements, components, and/or groups thereof.

The phrase “consisting essentially of” means that the scope of an aspect or embodiment is to be interpreted to encompass the specified materials or steps recited in the aspect or embodiment, including any materials or steps which do not materially affect the invention defined by the aspect or embodiment. Thus, the term “consisting essentially of when used in a claim of this invention is not intended to be interpreted to be equivalent to “comprising.”

“contacting” refers to causing two or more items to come into contact with each other. The items may be two or more of cells, a substrate, a fluid, and a purified mucin or biosimilar mucus, suitably as defined herein. Contacting may include causing or placing two or more of the above items into close physical relationship with each other.

“detecting”, “detect” and “detection” refer to the identification and/or quantification of a compound of interest (e.g., drug, agent, etc.) in a sample. In some embodiments, detecting comprises determining the absence or presence of a compound of interest in a sample. In some embodiments, detecting comprises quantifying a compound of interest in a sample. In some embodiments, detecting comprises identifying and/or quantifying a compound of interest in a sample at different time points. In some embodiments, detecting comprises identifying and/or quantifying a compound of interest in a first sample and in a second sample.

“absorption” or “perfusion” refers to the movement of a compound, for example a test compound or drug, into the bloodstream and through tissues following administration, as well as movement of a compound through a tissue model as described herein. Absorption or perfusion is determined by the compounds physicochemical properties, formulation, and route of administration.

“Tissue” as referred to herein is a group of cells which function together as a unit. A tissue is a level of cellular organisation between individual cells and a complete organ. Therefore, an organ is made up of multiple tissues. Tissue may be connective, muscular, nervous or epithelial. For the purpose of the present invention, the tissue may be epithelial, which secretes mucus.

The “epithelium” is a single layer of cells which covers all organismal surfaces which are exposed to the external environment, forming a semi-permeable barrier. Such surfaces include the airways, the skin or the digestive tract in a mammal.

The “gastrointestinal tract” refers to the complete system of organs and regions that are involved with ingestion, digestion, and excretion of food and liquids. This system generally consists of, but is not limited to, the mouth, oesophagus, stomach and or rumen, intestines (small and large), cecum (plural ceca), fermentation sacs, and the anus.

“intestinal cells” refers to cells that make up the mammalian intestinal epithelium. These may be both in the small intestine and colon. The mammalian intestinal epithelium of the gastrointestinal tract has a well-defined organizational structure. The epithelium can be divided into two regions, a functional region that houses differentiated cells (villi-coated absorptive enterocytes) and a proliferative region (crypts of Lieberkuhn) that represents the epithelium stem cell niche. Multipotent epithelium stem cells reside in the crypts and give rise to four principal epithelial lineages: absorptive enterocytes, mucin secreting goblet cells, peptide hormone secreting enteroendocrine cells, and Paneth cells.

The “intestine” refers to the mammalian small intestine and mammalian large intestine.

The term “in vitro” refers to processes performed or taking place outside of a living organism. In some embodiments, the processes are performed or take place in a culture dish.

The term “in vivo” refers to processes that occur in a living organism.

The term “mucin” refers to a high molecular weight, glycosylated protein, which is a component of mucus and which has the ability to form mucin-mucin interactions to form a gel. Mucin-mucin interactions include covalent and non-covalent bonding. Covalent bonding includes disulphide bonds between mucin glycoproteins.

The term “mucus” refers to an aqueous colloid, comprising mucin and optionally inorganic salts, immunoglobulins, nucleic acids, proteins, and/or glycoproteins. Native mucus is swellable. Native mucus moistens and protects tissue, but may also be permeable to small compounds like amino acids and small sugars, particularly in the small intestine. Native mucus is a viscid secretion, usually rich in mucins and is produced by mucous membranes which it moistens and protects.

“tissue explant” refers to an isolated piece or pieces of tissue.

A “substrate” as referred to herein is any surface, which is suitable for supporting biological material, for example a cell, cell population, cell culture, tissue, or a fluid or other biological material or composition, for example as described herein. A surface may be suitable for hosting a process or reaction, for example, growth and development of a cell or organism, cell population, cell culture, or tissue.

A “gel” is a semi-solid, jelly like substance, which does not flow when in the solid state. A gel comprises a 3D cross linked network which provides the gel is semi-solid structure. A gel may be a hydrogel, which comprises a network of insoluble but hydrophilic polymer chains. A gel may be defined in terms of its viscoelasticity or rheological properties.

A “model” as referred to herein is a three dimensional representation of all or part of a native tissue structure.

A “buffer” is s solution which can resist significant changes in pH upon the addition of small amounts of acid or alkali. A buffer is a mixture of a weak acid and its conjugate base, or a weak base and its conjugate acid.

“Dialysis” is the process of separating molecules in solution, based upon the difference in their ability to diffuse through a semi-permeable membrane. Dialysis may be used for the removal of small molecules such as salts, peptides and dyes from larger molecules such as DNA, proteins (including glycoproteins), and polysaccharides. A semi-permeable membrane may be cellulose, modified cellulose or a synthetic parameter. The process may require the use of a dialysis buffer, into which the sample to be dialysed is placed. Optimisation of the dialysis process may require adjustment of parameters based on sample volume, size of molecules being separated, the type of membrane, and the geometry of the membrane. Suitable conditions will be known to persons skilled in the art.

“Fractionation” is a separation process in which a mixture is divided into a number of smaller fractions, based upon a specific property of the individual components of the mixture. An example of fractionation is centrifugation.

As used herein, “cell viability refers to the ability of a cell to remain metabolically active for growth and function.

A “test compound” is any compound to be investigated, for example for use in the human or animal body.

The “LVE region” indicates the range in which the test can be carried out without breaking down or disrupting the structure of the sample.

By the “purified” herein is meant a mucus which has been subject to a process which removes some or all of the components other than mucin, such as nucleic acid, protein, immunoglobulins, salts, enzymes, and contaminants. Therefore a purified mucus comprises a higher concentration of mucin compared to the native mucus from which it is derived. A purified mucus may comprise a lower concentration of one or more of nucleic acid, protein, immunoglobulins, salts, enzymes, and contaminants compared to the native mucus from which it is derived. A purified mucus may be partially purified mucin, or fully purified mucin, for example where the product is substantially pure mucin (i.e at least 95% mucin or more).

“Biocompatible” may also be referred to as “biologically compatible”, and herein means that the substance or substrate to which it refers is substantially not toxic or harmful to a living cell.

The term “reconstituted mucus” as used herein refers to a purified mucus of the invention which has been reconstituted in a solution such as a buffer. A reconstituted mucus is suitably biocompatible.

### Mucus Preparation

The present invention is based upon purifying mucus, to provide a preparation comprising mucin wherein the preparation shares rheological properties of the native mucus, and can form a gel without the requirement for synthetic or non-native polymers. The method of the present invention is able to purify mucus to provide a preparation in which the native mucin is not denatured, degraded or hydrolysed, and therefore retains mucin: mucin interactions. It is these interactions which provide the mucus preparation with its viscoelastic properties and ability to form a gel. The purified mucus of the invention may share one or more rheological properties with a native mucus, suitably the native mucus from which it is derived. The purified mucus of the invention has also surprisingly been shown to be biocompatible, and therefore be capable of supporting live cells. Thus, the present invention provides a purified, biocompatible mucus according to the first aspect. A purified, biocompatible mucus of the present invention may be prepared using a method of the third aspect.

In an embodiment, the purified, biocompatible mucus is obtained from a mucus sample, for example from a tissue sample. Suitably, the purified, biocompatible mucus is obtained from mucus obtained from a tissue sample, by a method according to the third aspect of the invention, as described herein.

The mucin of the purified biocompatible mucus may be a polymer. A mucin polymer may have a molecular weight of 1 to 10 million Daltons. Mucin may be a glycoprotein. A mucin glycoprotein may comprise a peptide core, made up of multiple mucin monomer units linked by disulphide bonds, attached to multiple carbohydrate side chains. The carbohydrate side chains may be O-linked or N-linked oligosaccharides. One or more carbohydrate side chains may be negatively charged. The mucin may be saturated with O or N linked oligosaccharides. Mucin may comprise cysteine rich, lightly glycosylated amino- and carboxy-terminal regions. Mucin may be a transmembrane protein, comprising a cytoplasmic region, a transmembrane region, and an extracellular glycoprotein.

The purified, biocompatible mucus is obtained of the present invention may form a gel when placed under suitable conditions, of pH, concentration, temperature etc required for gelation. Such conditions will be known or can be derived by a skilled person based on his knowledge of the art. Suitably, a purified, biocompatible mucus of the present invention will have substantially the same viscoelastic properties as the mucus from which it is derived, when both are placed under the same conditions.

In particular, a purified, biocompatible mucus of the invention may exhibit both liquid like and solid like properties. A purified, biocompatible mucus of the invention may exhibit linear viscoelasticity. A purified, biocompatible mucus of the invention may have shear strain and stress strain in the linear viscoelastic region which is substantially the same, or greater than, that of the native mucus from which it is derived, or from a cleaned version of the native mucus from which it is derive. A purified, biocompatible mucus of the invention may have a shear strain in the Linear viscoelastic region greater than 5%. A composition of the invention may have a storage modulus (G′) of 1 or more. A composition of the invention may have a loss modulus (G″) of 1 or more. A composition of the invention may have a phase angle within the Linear viscoelastic region of at least 3 to 20, or any range of integer therebetween. Suitably, a mucus preparation of the present invention may share one or more, two or more or three of more rheological properties with native or cleaned mucus, selected from the group of shear strain, stress strain, storage modulus, loss modulus and phase angle.

A purified biocompatible mucus suitably has one or more physical and/or chemical properties which resemble native mucus, in addition to the viscoelastic properties as defined above, for example permeability, charge, and containing region-specific mucins.

The purified biocompatible mucus of the invention may comprise one or more additional components, including but not limited to lipid, salts, immunoglobulins, enzymes, proteins, nucleic acid (DNA or RNA), glycoprotein, cells, and water. One or more of such components may be native or non-native to the mucus from which the purified biocompatible mucus is derived.

In an embodiment, a purified biocompatible mucus of the invention does not substantially comprise lipid, nucleic acid, or protein of the native mucus. Thus, the mucus preparation of the invention is prepared by a method which removes substantially all of the lipid, protein and nucleic acid from the native mucus. The purified mucus of the present invention may have a density of 1.3 to 1.6 g/mL, suitable 1.4-1.5 g/mL, most suitably 1.42 g/mL. Suitably, the biocompatible mucus does not comprise components (for example lipid, nucleic acid, or protein) having a density above or below 1.3-1.6 g/mL. Optionally, a purified mucus of the present invention does not comprise components having a molecular weight below 30 kDa, preferably 20 kDa, or preferably 15 kDa. The density and/or molecular weights may be provided in relation to a purified mucus after the dialysis and/or equilibrium density gradient centrifugation, but prior to addition of any other components such as a buffer.

In an embodiment, a purified biocompatible mucus of the invention does not comprise components having a molecular weight of 100 kDa or less, preferably 70 kDa or less, preferably 60 kDa or less, or preferably 50 kDa or less.

By biocompatible means that the purified mucus is not toxic or detrimental to a cell. Therefore, the purified mucus is compatible with a cell, such that it does not induce or cause cell death, within a suitable time period. The composition may be capable of sustaining the viability of a cell. Therefore, the composition does not cause substantial loss of cell viability. By substantial is loss of viability of 30% or more of the cells in a population, over a defined period of time for example several minutes, one or more hours, or up to multiple days, depending upon the nature of the composition and the cell type, and the conditions of the model.

In an embodiment, the mucus is purified from mammalian or non-mammalian mucus. A mammal may be a human; a domestic animal such as a dog, cat, guinea pig, rabbit, rat, mouse; a farm animal such as a pig, sheep, goat, donkey, horse, cattle, cow; or any other large mammal for example a deer, antelopes, elephants, camels, llamas and so on. Any other suitable mammal from which mucin may be extracted will be known to persons skilled in the art. The mammal may be a pig. The mammal may be a human. A non-mammal may be a fish, amphibian, snail, slug, or any other suitable animal. Suitably, the purified biocompatible mucus is derived from a mammal, suitably an animal, suitably a human. Suitably, the purified biocompatible mucus is derived from a pig, suitably from gastro-intestinal system of a pig, suitably from the intestine (large or small) or stomach of a pig.

The purified biocompatible mucus may be extracted from any suitable tissue of the mammal or non-mammal. Where the purified biocompatible mucus is extracted from a mammal, it may be extracted from any tissue which naturally comprises or secretes mucus. For example, the tissue from which mucin is extracted according to the present invention may be gastro-intestinal tissue, including but not limited to stomach, duodenum, oesophagus, buccal, lingual or colon tissue; airway tissue including but not limited to the lungs, trachea, bronchi and bronchioles, and mouth; female reproductive tissue including but not limited to cervical tissue and vaginal tissue; and nasal tissue. In an embodiment, the tissue from which the biocompatible mucus is extracted may comprise epithelial cells. Where the biocompatible mucus is extracted from a non-mammal, it may be extracted from the external surface of the non-mammal.

Purified biocompatible mucus as referred to herein may be 100% pure mucin, or may comprise other non-mucin components, for example contaminants, components of native mucus as described herein, including biological molecules remaining from the extraction process. Purified mucus as referred to herein may comprise 10%, 20%, 30%, 40%, 50%, 60%, 70%, 75% 80%, 85%, 90%, 91%, 92%, 93%, 94%, 95%, 96%, 97%, 98% or 99% mucin. Suitably, the purified biocompatible mucus comprises at least 35%, suitably at least 40% mucin. The remainder of the preparation may include water, salts, lipids, proteins, sugars, glycoproteins, immunoglobulins, bacteria, reagents, and/or contaminants. Where the purified biocompatible mucus of the present invention is produced by a method of the third aspect, the purified biocompatible mucus may comprise 10%, 20%, 30%, 40%, 50%, 60%, 70%, 75% 80%, 85%, 90%, 91%, 92%, 93%, 94%, 95%, 96%, 97%, 98% or 99% mucin, prior to reconstitution of the purified mucus in a buffer. Suitably, the purified biocompatible mucus comprises at least 35%, suitably at least 40% mucin. Mucin is rich in serine, proline and threonine. A purified biocompatible mucus of the present invention may be rich in serine, proline and threonine. For example, a purified biocompatible mucus of the present invention may comprise at least may comprise 10%, 20%, 30%, 40%, 50%, 60%, 70%, 75% 80%, 85%, 90%, 91%, 92%, 93%, 94%, 95%, 96%, 97%, 98% or 99% serine, proline and threonine, most suitably at least 70%, 75% 80%, 85%, 90%, 91%, 92%, 93%, 94%, 95%, 96%, 97%, 98% or 99% serine, proline and threonine. A purified biocompatible mucus of the present invention may substantially lack, or contain low (e.g less than 30%, 20%, 15%, 10%, 9%, 8%, 7%, 6%, 5%, 4%, 3%, 2% or 1%) amino acids other than serine, threonine or proline, such as valine,

Suitably, the purified biocompatible mucus of the present invention may comprise less than 1%, 2%, 3%, 4%, 5%, 6%, 7%, 8%, 9%, 10%, 15%, 20%, 25%, 30%, 40%, 50%, 60%, 70%, 80% or 90% impurities (i.e non-mucin).

The purified biocompatible mucus referred to herein may be a viscous, gel-like preparation. Alternatively it may be a solid, for example, it may be dried or lyophilised to provide a preparation for storage. Alternatively, it may be a liquid, for example following reconstitution in a suitable buffer or dilution.

The purified biocompatible mucus may be provided in a buffer, to form a composition. A buffer may be suitable for use with cells or live tissue. A buffer may be suitable for use with acidic gastro-intestinal secretions. A buffer may not alter the viscoelastic properties of the purified biocompatible mucus. A buffer may be cell compatible. Examples of suitable buffers include, but are not limited to, deionized water, a phosphate buffer (for example phosphate buffered saline), HEPES buffer, MOPS buffer, MES buffer, Tricene, Bicene, TAPS, ACES, MOPSO and BES. Other suitable buffers may be used and will be known to a person skilled in the art. In an embodiment, the buffer is phosphate buffered saline, which represents bodily fluids at the correct pH and osmolarity. The buffer may be supplemented, for example with calcium and/or magnesium. The buffer may be PBS supplemented with calcium and magnesium.

The purified biocompatible mucus may be provided in a buffer at any suitable concentration. In an embodiment, the concentration of purified biocompatible mucus in the composition is similar to that of a native mucus. The purified biocompatible mucus may be present in the composition at 2, 3, 4, 5, 10, 15, 20, 25, 30, 35, 40, 45, 50 mg/ml or more. In an embodiment, the biocompatible mucus is present in the composition at a concentration of 5-120 mg/ml, suitably 30-100 mg/ml, suitably 40-80 mg/ml. The concentration will depend on the presence and amounts of other components in the composition as described herein.

The purified biocompatible mucus may be provided in combination with a cell, or as a composition comprising a cell.

The mucin present in the purified biocompatible mucus may be human MUC2, MUC5AC, MUC5B, MUC6, MUC19, MUC7 or a non-human equivalent thereof, including for example, ovamucin (egg white), spiggin (MUC19 derived mucin from sticklebacks), SCO-spondin, or otogelin (see also Lang et al Mol Biol Evol. 2016 August: 33 (8): 1921-1936.) In an embodiment, the mucin is MUC5AC or MUC5B. In an embodiment, the mucin is MUC2.

In an embodiment, there is provided a composition consisting essentially of purified biocompatible mucus of the first aspect, and a buffer. The biocompatible mucus and the buffer may be as described herein. The biocompatible mucus may be purified according to the third aspect of the present invention, as described herein. The concentration of the mucin may be 20-120 mg/ml, suitably 30-100 mg/ml, suitably 40-80 mg/ml, suitably 50 mg/ml.

### Tissue Model

In a second aspect of the present invention, there is provided a tissue model comprising i) a cell population and ii) purified biocompatible mucus according to the first aspect. Suitably, the purified mucus is in contact with the cell population. A tissue model according to the second aspect of the invention may be useful in a screening assay, for example for drug absorption, drug dissolution, and drug toxicity. The second aspect of the invention is based upon the surprising finding that the purified biocompatible mucus of the first aspect can be used to build a reconstituted mucus layer which can be combined with a cell population, in a manner that does not kill the cells. Such a model provides a robust and physiologically relevant in vitro method in which pharmaceutical formulations can be screened, and used to improve and inform human studies.

A tissue model according to the second aspect of the invention may comprise a substrate comprising a cell support portion. The cell population may be in contact with the cell support portion of the substrate. The cell population may form a layer on the cell support portion.

A tissue model may be described as having a basolateral surface which is the bottom of the substrate, and an apical surface which is the top surface of the tissue model. In a tissue model of the present invention, purified biocompatible mucus is applied to the top surface of the cell population, in contact with the cell population. The purified mucin may not be in direct contact with the cell support portion of the substrate.

In an embodiment, there is provided a tissue model of the first aspect, comprising in discrete layers:
i. a substrate comprising a cell support portion;
ii. a cell population in contact with the cell support portion of the substrate;
iii. purified biocompatible mucus, in contact with the cell.

Herein, any suitable substrate having a portion thereof suitable for hosting a cell population, may be used. Suitably, the substrate is suitable for hosting, or culturing, mammalian cells. A substrate may be one which is easily sterilized and is biocompatible. Suitable substrates will be known to persons skilled in the art, and may include but are not limited to glass, carbon, an organic material (e.g. cotton), nitrocellulose, dextran, gelatin, polymeric material (e.g. plastic, poly(methylmethacrylate, polyamides, polyesters; polystyrene; polypropylene, polyurethane polylacrylates, polyvinyl compounds (e.g., polyvinylchloride); polycarbonate; polytetrafluoroethylene (PTFE, Teflon), acrylic copolymer polyglyolic acid (PGA)) fluoropolymers: fluorinated ethylene propylene; polyvinylidene; polydimethylsiloxane), metals (for example gold, silver, aluminium, titanium, stainless steel), silicon substrates (fused silica, polysilicon, or single silicon crystals).

A substrate may be rigid or may be elastic. A substrate may be porous, or may be non-permeable. A material which is not naturally porous can be made porous by methods available to the skilled person, for example sintering, etching, leaching, lithography, or laser micromachining.

A substrates may be a slide, chip, plate, flask, vial, film, microstructure (including a groove, well or post), multi-well plate, dual chamber plate, or any other suitable form. A substrate can be in any suitable shape or configuration, including flat, tubular, curved, spherical, ellipsoid, etc., including composites (e.g., to emulate macroanatomical structures). A substrate may comprise a planar surface, upon which a cell population may be provided. A substrate can be provided or mounted on a porous carrier (e.g., a porous membrane, a mesh, an inorganic grid, a hydrogel, or a combination thereof) to lend structural support thereto. A substrate may be two- or three-dimensional or any combination thereof.

Suitably, the substrate may be a dual chamber plate, wherein each well comprises a lower chamber and an upper chamber. The upper chamber may sit on top of, or be placed within, the lower chamber. Suitably, the upper and lower chambers are substantially aligned. The lower chamber may be referred to as a basolateral chamber, and the upper chamber may be referred to as an apical chamber. The bottom surface of the upper chamber may be permeable, to allow fluid to pass from the upper chamber to the lower chamber. Suitably, the upper chamber sits fully or partially within the lower chamber, to allow for fluid communication between the two chambers. The upper chamber may comprise the cell population and purified mucin layers. The lower chamber may comprise a buffer or suitable cell culture medium to maintain the cell population. Thus, for example in a small intestine model, the lower chamber represents the basolateral side of the epithelial cells, for villi associated circulatory blood and lymph vessels. The upper chamber represents the lumen of the small intestine.

Suitably, a substrate may be provided in an anaerobic envelope, to provide an anaerobic environment. Where the substrate is a dual chamber plate as described above, the lower chamber may be provided with an air supply to create an aerobic environment, whereas the upper chamber may be an anaerobic environment.

Selection of a suitable substrate may depend upon the type of cells to be used in the model. Factors may include the ability of the cells to adhere to the substrate, and the effect of the substrate on the cells.

A substrate may be modified to allow or improve adherence of cells thereto. All or part of the substrate may be modified. Suitable modifications will be known to persons skilled in the art, and may include for example application of a protein (e.g. collage or fibronectin), extracellular matrix or components thereof, sugars, proteoglycans to the surface of the substrate.

The substrate may be single well, or may be a multi-well plate. A multi-well plate may comprise any suitable number of wells, for example 6, 12, 24, 48, 96, 384 or 1536 wells. The wells may be microwells.

In a model of the second aspect, a cell population is placed in contact with the substrate, suitably a planar surface of a substrate such as the bottom of a well which forms the cell support portion. A cell population may comprise one or more cells, suitable two or more, up to 1000, 10,000, 1×10⁸, 2×10⁶, 3×10⁶ or more.

A cell population may form a layer of cells. Suitably, the cell population forms a layer of at least 50, 60, 7, 80, 90 or 100% confluency. Thus, the cell layer may be referred to as being confluent. A layer of cells in contact with the cell support portion may be a monolayer of cells (meaning that the layer is one cell deep), or more than one layer of cells (two or more cells deep). The cells may comprise one type of cell, or two or more different cell types. In a multi-layer cell population, each layer may substantially comprise a different cell type. The cell population provided on the substrate may be a tissue explant. A tissue explant may be a single layer of tissue or may be two or more layers of tissue. A tissue explant may be placed onto a cell support portion of a substrate and compressed. A tissue explant may be derived from an organism, such as a mammal, or may be cultured in vitro.

A tissue model may have the architecture of the corresponding native in vivo tissue.

Where the cells form a layer on the cell support portion of a substrate, they layer may be flat or may be in the folded or formed into a three dimensional shape, for example to mimic a tissue structure, such as the crypt structure or crypt-villus structure of in vivo intestines, or the vaginal stratified squamous epithelial layers.

A cell population, optionally as a tissue explant, may be derived from gastro-intestinal tissue, airway tissue, nasal tissue, optic tissue, or female reproductive tissue. A cell population, optionally as a tissue explant, may be derived from the ileum, jejunum, stomach, duodenum, oesophagus, buccal, lingual or colon of the gastrointestinal tract, or from the nose, lung, bronchi, bronchioles, or mouth; or from the cervix or vagina, or from the eye. The cells may be obtained from a mammal as defined herein, for example a pig. The cells may be derived from a human. In an embodiment, the tissue explant is derived from the gastro-intestinal tract of a human, suitably from the ileum, jejunum, stomach, duodenum, oesophagus, buccal, lingual and/or colon of a human. In an embodiment, cells for use in a model of the second aspect are mouth cells, stomach cells, small intestine cells, large intestine cells, nasal cells, optic cells, vaginal cells, cervical cells, or lung, or bronchial, or other airway cells. The cells may be epithelial cells. The cell culture may be derived from a cell line. In an embodiment, the cell culture is a caco-2 cell culture or a calu-3 cell culture.

The cell population or tissue may comprise native fluids or tissue components, for example the phlegm, nasal mucus, urine, vomit, bile, blood, vaginal discharge, semen, or other biological discharge, microorganisms such as bacteria, and cell debris.

A cell population may be placed in contact with a cell support portion of a substrate, in a manner which maintains the native cell polarity. Therefore a native basal surface of the tissue may be placed in contact with the cell support portion of the substrate, and a luminal surface of the tissue (which in vivo would face the lumen of a tissue or organ is uppermost), and is separated from the cell support portion of the substrate by the body of the cell population.

The top surface of a tissue model may be referred to herein as an apical surface, and the bottom surface may be referred to as a basolateral surface.

A substrate may comprise two or more tissue explants. Where the substrate is a multi-well plate for example, each well or cell support portion may be in contact with a different tissue explant.

The explants may be the same or different tissue types. The explants may be from the same type of mammal or different types of mammal. The tissue explants may all be human.

The cell population of the model of the second aspect of the invention may be maintained in culture, for example for any suitable time period, for example from several minutes to an hour or more, 30 minutes to 2 hours, 30 minutes to 3 hours, 30 minutes to 4 hours, 30 minutes to 5 hours, 30 minutes to 6 hours, 1 to 12 hours, 1 to 24 hours, 1 to 36 hours, 1 to 48 hours, 1 to 72 hours or 1 hour to 1 week, 2 weeks, 3 weeks, 4 weeks or more, under suitable conditions. The cell population may require an exogenous growth factor to be maintained in culture, or may not require an exogenous growth factor. By the term “maintained in culture” is meant that the cell population is maintained under conditions suitable for cell growth and/or survival, in vitro. This may require the provision of essential nutrients (e.g., amino acids, carbohydrates, vitamins, minerals), growth factors, hormones, gases (e.g., 02, C02), and maintenance of an appropriate physicochemical environment (e.g., pH, osmotic pressure, temperature). The conditions may be aerobic or anaerobic. Examples of substances which may be provided in a cell culture to maintain cell growth and/or survival include but are not limited to: fibronectin; foetal calf/bovine serum; laminin; epidermal growth factor (EGF); R-spondin; noggin; cytokines (e.g., interleukin (e.g., IL-6, IL-17, IL-22), tumor necrosis factor (TNF)); ephrin receptors (e.g., EphrinB, EphBs); bone morphogenetic proteins (BMPs, BMP-2, BMP-7); Wnt (wingless-related integration site) (e.g., Wnt3, Wnt3A, and other Wnts); notch signaling factors (notch receptors); DIII/4; Noggin; Greml; Grem2; acetate; butyrate; proprionate, desaminotyrosine, catecholamine (e.g., dopamine, norepinephrine) cytokines, and/or short chain fatty acids.

The purified biocompatible mucus may be as defined in the first aspect. Therefore in an embodiment the purified biocompatible mucus may be a composition comprising or consisting essentially of purified biocompatible mucus and a buffer as described in the first aspect.

The purified biocompatible mucus may be provided as a layer, on the cell. The layer may be a uniform layer or a non-uniform layer. The layer may substantially cover all of the cells. The layer may be of any suitable thickness. Suitably, the purified biocompatible mucus layer mimics the thickness of mucin found in a native tissue. The thickness of the mucin layer may be any suitable thickness depending upon the purpose of the model, the conditions, and the drug to be tested. A suitable thickness may be 500 μm to 15 mm, 400 μm to 15 mm, 300 μm to 15 mm, 200 μm to 15 mm, 100 μm to 15 mm, 10 μm to 15 mm, 1 μm to 15 mm, 0.1 mm to 15 mm, 0.5 mm to 15 mm, 1 mm to 15 mm, or 3 mm to 15 mm, or any range using the upper or lower limit of any of the aforementioned ranges, or any integer which is the upper or lower limit or falls within the aforementioned ranges.

The tissue model as described herein may additionally comprise a fluid. The fluid may be provided on the apical surface of the mucin layer. The fluid therefore may not be in direct contact with the cell. The fluid may be provided to mimic the native tissue environment, and may be for example a digestive fluid, saliva, vaginal secretion, tears, or airways secretions, or a microbial cell population. The fluid may be a native fluid extracted from a mammal as described herein, or may be a synthetic fluid. The fluid may be an end-point digestive fluid (EPDF), for example a fluid comprising salivary, gastric, and small intestinal fluid, optionally bile and/or enzymes, for example pepsin and/or pancreatin. A suitable end-point digestive fluid is shown in Table 1. The compositions of the diluents of the EPDF are shown in Table 2. Any suitable volume of fluid may be applied.

TABLE 1
Component ratios for those involved in the preparation of EPDF

| Component type | Volume (ml) |
|---|---|
| Salivary Diluent | 10 |
| Gastric Diluent | 80 |
| Small Intestinal Diluent | 60 |
| Porcine Bile | 25 |

| Enzyme Type | Amount (mg) |
|---|---|
| Pancreatin | 40 |
| Pepsin | 420 |

TABLE 2
Components of the three diluents involved in EPDF preparation.

| Diluent Type | Components |
|---|---|
| Salivary Diluent (pH: 7.4) | NaHCO₃ (62 mM; 5.21 g/L) |
| | K₂HPO₄•3H₂O (6 mM; 1.045 g/L) |
| | NaCl (15 mM; 0.88 g/L) |
| | KCl (6.43 mM; 0.43 g/L) |
| | CaCl•2H₂O (3 mM; 0.44 g/L) |
| Gastric Diluent (pH: 2) | Urea (5 mM; 0.3 g/L) |
| | K₂HPO₄ (2 mM; 0.27 g/L) |
| | NaCl (49.6 mM; 2.9 g/L) |
| | KCl (9.4 mM; 0.7 g/L) |
| SI Diluent (pH: 8) | NaHCO₃ (110 mM; 9.24 g/L) |
| | K₂HPO₄•3H₂O (2.5 mM; 0.44 g/L) |
| | NaCl (54.9 mM; 3.21 g/L) |
| | CaCl·2H2O (1 mM; 0.15 g/L) |
| | Urea (1.67 mM; 0.1 g/L) |

The three or more layers of the tissue model may be stacked such they are fully or partially aligned with one or more layers of the model. fully or partially overlap. Suitably, two or more of the layers fully align. Suitably, all of the layers fully align.

The present invention may provide a model of any mucus covered epithelial tissue.

In an embodiment, a tissue model of the second aspect of the invention is a gastric model. In an embodiment, a gastric tissue model comprises in discrete layers: i) a basal substrate comprising a cell support portion; ii) a cell population in contact with the cell support portion of the substrate; preferably wherein the cell population is a Caco-2 cell population; iii) purified biocompatible mucus, in contact with the cell; and optionally iv) gastric juice.

In an embodiment, a tissue model of the second aspect of the invention is a small intestinal model. In an embodiment, a small intestinal tissue model comprises in discrete layers i) a basal substrate comprising a cell support portion; ii) a cell population in contact with the cell support portion of the substrate; preferably wherein the cell population is a Caco-2 cell population; iii) purified biocompatible mucus, in contact with the cell; and optionally iv) gastro-intestinal fluid.

In an embodiment, a tissue model of the second aspect of the invention is a large intestinal model. In an embodiment, a large intestinal tissue model comprises in discrete layers i) a basal substrate comprising a cell support portion; ii) a cell population in contact with the cell support portion of the substrate; preferably wherein the cell population is a Caco-2 cell population; iii) purified biocompatible mucus, in contact with the cell; and optionally iv) gastro-intestinal fluid, preferably an end-point digestive fluid as described herein.

In an embodiment, a tissue model of the second aspect of the invention is an airway tissue model. In an embodiment, an airway tissue model comprises in discrete layers i) a basal substrate comprising a cell support portion; ii) a cell population in contact with the cell support portion of the substrate; iii) purified biocompatible mucus, in contact with the cell; and optionally iv) saliva, alveolar fluid, phlegm. The cell population may comprise any suitable airway cells.

In an embodiment, a tissue model of the second aspect of the invention is a female reproductive tissue model. In an embodiment, a female reproductive tissue model comprises in discrete layers i) a basal substrate comprising a cell support portion; ii) a cell population in contact with the cell support portion of the substrate; iii) purified biocompatible mucus, in contact with the cell; and optionally iv) vaginal fluid. The cell population may comprise any suitable cells of the femal reproductive system, including for example vaginal cells, cervical cells, uterine cells, fallopian tube cells, or ovarian cells.

In an embodiment, a tissue model of the second aspect of the invention is a optic or nasal tissue model. In an embodiment, an optic or nasal model comprises in discrete layers i) a basal substrate comprising a cell support portion; ii) a cell population in contact with the cell support portion of the substrate; iii) purified biocompatible mucus, in contact with the cell; and optionally iv) nasal discharge or tears, respectively. The cells may be any suitable cells of the optic or nasal system.

In an embodiment, the present invention provides two or more models placed in series, to provide a gastro-intestinal model. A gastro-intestinal model may comprise two or more of an airway model, gastric model, small intestine, and large intestine model in series.

In an embodiment, a model of the present invention may comprise one or more test compounds, for example a pharmaceutical agent. A test compound such as a pharmaceutical agent may be applied to the model to asses permeability of the agent.

In any of the tissue models provided herein, the purified biocompatible mucus may be reconstituted in a solution, such as a buffer, prior to use in the tissue model. The buffer may be any suitable buffer, for example PBS. A suitable buffer may be biocompatible. Any suitable method of reconstitution may be used, which does not substantially disrupt mucin: mucin interactions. A reconstituted mucus of the invention will suitably be biocompatible.

### Method of Purifying Mucus

The present invention provides a method of purifying mucus, wherein the method comprises removal of substantially all lipids, nucleic acids, and/or proteins, and/or any molecules below a pre-determined size, suitably 100 kDa, more suitably 70 kDa, wherein the step of removal comprises dialysis and/or equilibrium density gradient centrifugation. Suitably, the method does not comprise denaturation, degradation or hydrolysis of mucin. Optionally, the method may comprise a further separation step, for example centrifugation, to remove components of native mucus, contaminants, excess water and/or reagents. Suitably, a method of the invention provides a purified biocompatible mucus of the first aspect.

In an embodiment, the present invention provides a method of purifying mucus, wherein the method comprises i) providing a sample comprising mucus; ii) solubilising any mucin present in the sample; iii) separating from the mucus any nucleic acids, proteins, lipids; and iv) removing any contaminants, undesirable components or reagents from the mucus; to provide a purified, biocompatible mucus. Step iii) may comprise equilibrium density gradient centrifugation. Step iv) may comprise removing any cytotoxic components, reagents, and/or enzymes, and may comprise dialysis of the mucus. Suitably, the method comprises separating from the mucus a fraction having a density gradient of 1.3 to 1.6 g/mL.

Solubilisation may be performed in order to increase the solubility of any mucin in the sample, using any suitable method. Suitable methods may include but are not limited to incubation of the tissue sample with a chaotropic agent (such as urea, thiourea, in particular 8M urea, or a combination of 2M urea and 5-8M thiourea), ionic, non-ionic or zwitter-ionic detergent (such as SDS, NP-40, Triton X, CHAPS, and sulphobetaines), carrier ampholyte, or reducing agent (such as DTT or TBP). After solubilisation, any insoluble material may be removed, for example by centrifugation.

The step of separating from the mucus any nucleic acids, proteins, lipids may suitably comprise equilibrium density gradient centrifugation, and/or dialysis. The method may comprise equilibrium density gradient centrifugation to isolate a fraction having a density of 1.3-1.6 g/mL, suitably 1.4-1.5 g/mL, or most suitably 1.42 g/mL, or which is identified as having the highest proportion of mucin glycoprotein and lowest proportion of nucleic acid, or lipid or protein, may be selected using methods available to the skilled person, for example periodic schiff's acid analysis and analysis of OD260/OD280. Alternatively, dialysis may be used to remove any molecules having a molecular weight below 100 kDa, suitably 70 kDa, most suitably 50 kDa. A combination of equilibrium density gradient centrifugation and dialysis may be used to provide a purified mucus which substantially lacks nucleic acid, lipid and protein, and suitably also any chemical reagents or enzymes. Suitable combinations of centrifugation and dialysis may be identified by the skilled person, to provide a purified mucus which has a low nucleic acid content and a high glycoprotein content, or a density of between 1.3-1.6 g/mL or lacking molecules below 100 kDa, suitably 70 kDa, most suitably 50 kDa. Where ultracentrifugation is used, suitably, a caesium salt gradient is used, suitably caesium chloride. It is envisaged that other suitable methods may be used, which have the effect of substantially purifying or separating mucin from mucus. Such methods such as fractionation, centrifugation etc will be known and available to persons skilled in the art.

Suitably, the step of separating any nucleic acids, proteins, lipids or molecules from the mucus may comprise removal of any such components which have a density above or below 1.3-1.6 g/mL or which are below 100 kDa, more suitably below 70 kDa. Any suitable combination of ultracentrifugation and/or dialysis may be used to provide a mucus preparation having a density of 1.3-1.6 g/mL or from which molecules below 100 kDa, suitable 70 kDa, more suitably 50 kDa have been removed.

A method may comprise removal of any unwanted components or chemicals, such as reagents, enzyme inhibitors etc, may be removed. Any suitable method may be used, for example dialysis. Suitably, the method may comprise dialysis to remove any chemicals, contaminants, or reagents. Suitably, the dialysis may have a Molecular Weight Cut Off (MWCO) of 30 kDa or less, 25 kDa or less, 20 kDa or less of 15 kDa or less. Any suitable semi-permeable membrane may be used, including but not limited to, of polysulfone, polyethersulfone (PES), etched polycarbonate, cellulose or collagen dialysis tubing may be used. The dialysis may be performed for any suitable period of time, which will depend on the MWCO of the tubing. A suitable time period may be example from 1 hour of more. 3 to 5 hours, 5 to 10 hours, 5 to 12 hours, 12 to 14 hours, 36 hours, 48 hours, 72 hours, 1 week, 2 weeks, 3 weeks, 4 weeks or more, or any integer or range therebetween. Such dialysis may be performed on a fraction selected following ultracentrifugation of a mucus sample.

In an alternative method, a purified mucus may be prepared using size exclusion chromatography. The fraction containing mucin can be separated from the other components in mucus as described herein using size exclusion chromatography. Suitable columns will be known to a person skilled in the art, but may include a Sepharose 2B or 4B column. Mucin may be collected from the column void volume.

Suitably, the method may comprise homogenisation of a tissue sample. Homogenisation may take place prior to solubilisation, and prior to exposure to an enzyme inhibitor, Suitable methods for homogenisation include, but are not limited to, grinding, mincing, chopping, pressure changes, osmotic shock, freeze-thawing, and ultra-sound. The sample may be refrigerated to reduce or prevent protein damage.

Suitably, the method additionally comprises exposing the sample to one or more enzyme inhibitors. Suitably, the sample is exposed to one or more enzyme inhibitors prior to solubilisation. An enzyme inhibitor may have the effect of reducing or preventing mucin lysis by an enzyme present in the sample. Suitable enzyme inhibitors include protease inhibitors. The method of the present invention may comprise exposing the sample to one or more protease inhibitors. Suitably, the method may comprise exposing the sample to an inhibitor of an aspartic protease, a cysteine protease, a serine protease, a metalloprotease, a thiol protease, and/or a threonine protease. Suitable inhibitors or combinations of enzyme inhibitors will be known and available in the art. By way of non-limiting example. A suitable enzyme inhibitor may be aminoheaxanoic acid, EDTA, iodoacetamide, N-ethyl maelinide, benzamidine HCL, or pmsf. Suitably, a combination of two or more enzyme inhibitors may be used. A suitable enzyme inhibitor combination may comprise any two or more inhibitors, selected from aminoheaxanoic acid, EDTA, iodoacetamide, N-ethyl maelinide, benzamidine HCL, and/or pmsf. Other suitable enzyme inhibitors or combinations thereof will be known to a person skilled in the art. An enzyme inhibitor may be applied to the tissue sample in any suitable amount, for example at a v/v ratio of 1:1, 2:1, 3:1 or more (enzyme inhibitor: tissue sample by volume). Suitably, the enzyme inhibitor will not result in mucin degradation. The amount and concentration of an enzyme inhibitor may be selected to prevent degradation of mucin.

In an alternate embodiment, the method may comprise i) providing a sample comprising mucus; ii) dialysing the mucus to separate from the mucus any molecules under 100 kDa, suitably 70 kDa; and optionally iii) separating the mucus, for example by centrifugation.

In such an embodiment, a mucus sample may be dialysed using a dialysis tubing having a MWCO of 100 kDa or less, 70 kDa or less, 60 kDa or less, or 50 kDa or less. Any suitable semi-permaeable membrane may be used, including but not limited to, of polysulfone, polyethersulfone (PES), etched polycarbonate, cellulose or collagen dialysis tubing may be used. The dialysis may be performed for any suitable period of time, which will depend on the MWCO of the tubing. A suitable time period may be example from 1 hour or more, 3 to 5 hours, 5 to 10 hours, 5 to 12 hours, 12 to 14 hours, 36 hours, 48 hours, 72 hours, 1 week, 2 weeks, 3 weeks, 4 weeks or more, or any integer or range therebetween.

A method of this embodiment may further comprise separation, for example by centrifugation or any other suitable method, to remove excess liquid or other components of the mucus.

A method of the invention may comprise obtaining a mucus sample. A step of obtaining a mucus sample may comprise scraping or extracting mucus from a tissue sample. A mucus sample may be diluted prior to purification, and any suitable dilution, for example 1:50.

A method of the present invention may further comprise combining the purified mucus with a solution, for example a buffer. The buffer may be as described herein. This step may be referred to as reconstitution of the purified mucus.

A method of the present invention may comprise combining a purified biocompatible mucus of the invention with one or more components selected from lipid, salts, protein, nucleic acid (DNA or RNA), glycoprotein, cells, and water. The buffer and/or one or more of lipid, salts, protein, nucleic acid (DNA or RNA), glycoprotein, cells, and water may be as described herein. The method may further comprise placing the purified biocompatible mucus under conditions to allow gelation of the mucus.

A method of the present invention may further comprise drying, freezing, reconstituting or diluting a purified biocompatible mucus of the present invention. In a preferred embodiment, a purified mucus of the invention is lyophilised. In an embodiment, a lyophilised purified mucus of the invention is reconstituted in a suitable buffer, for use.

The present invention may also provide a method of preparing a purified biocompatible mucus according to the first aspect, comprising purifying mucus as described herein, and combining the purified mucus with a buffer and one or more of lipid, salts, protein, nucleic acid (DNA or RNA), glycoprotein, cells, and water to prepare a biocompatible mucus as described herein. The buffer and/or one or more of lipid, salts, protein, nucleic acid (DNA or RNA), glycoprotein, cells, and water may be as described herein.

A method of purifying mucus from a tissue may comprise one or more sterilisation steps. A sterilisation step may be performed during the method of purifying the mucus from the tissue, for example by the application of one or more anti-microbial agents during the process, such as during dialysis. A sterilisation step may be performed after purification, or after lyophilisation. A method of the invention may comprise one, two, three or more sterilisation steps. Any suitable sterilisation method may be used, for example temperature changes, and/or UV light mediated sterilization, or solvent sterilisation. A suitable solvent may include, but is not limited to, ethanol, methanol, or IPA. A solvent based sterilisation may include application of the solvent, mixing with the tissue, cells or purified mucus, or composition, and evaporation of the solvent. Any one or more of the purified mucus or composition comprising the purified mucus, may be sterilised.

The present invention may also provide a method of preparing a tissue model according to the second aspect, comprising purifying mucus as described herein, and applying the purified mucus to a cell population provided on a substrate. The method may comprise providing the purified mucus as a composition as described herein. The method of preparing a tissue model as described herein may further comprise applying a fluid as described herein to the purified mucus applied to the cell population.

Methods of forming a purified mucus may comprise employing one or more processes to encourage, allow or maintain gelation or the viscoelastic properties of the mucus. In an embodiment, a method of forming a purified biocompatible mucus may comprise one or more temperature changes, such as utilizing a heating and cooling cycle, to encourage gelation. In an embodiment, a method may comprise altering the pH to facilitate gelation of the purified biocompatible mucus. For example, raising and subsequently lowering the pH may encourage gelation. Any suitable number of temperature and/or pH raising and lowering cycles may be employed. In an embodiment, a method may comprise adding salt to the purified biocompatible mucus to encourage gelation of the mucin.

A method of preparing a purified biocompatible mucus may comprise storing the purified mucin. A method of storing may comprise lyophilisation of the purified mucin, composition or biosimilar mucus. Suitable conditions for lyophilisation will be known to a person skilled in the art.

### Uses of the Tissue Model

A purified biocompatible mucus or, a composition of the present invention or a tissue model of the present invention may be used in a number of applications, including but not limited to drug absorption testing, drug formulation development, profiling GI damage/side effects, nutrient uptake analysis, drug-microbiome interaction studies, in particular in the large intestine model, profiling intestinal damage, and pre/pro biotic testing.

Therefore, the present invention provides a method for determining or predicting absorption of a test compound through a tissue model wherein the tissue model comprises i) a substrate comprising a cell support portion; ii) a cell population in contact with the cell support portion of the substrate; and iii) purified biocompatible mucus, in contact with the cell; and wherein the method comprises contacting the purified biocompatible mucus with the test compound, and detecting the movement of the test compound in the tissue model; thereby determining the ability of the test compound to traverse the purified biocompatible mucus and cellular layers of the tissue model. The presence of the test compound at the substrate indicates the ability of the compound to be absorbed through the tissue model.

The tissue model may comprise purified biocompatible mucus or a composition comprising the purified biocompatible mucus as described in the first aspect of the invention. The tissue model may be as described in the second aspect of the invention.

The step of detecting the movement of the test compound in the tissue model may comprise determining the concentration of the test compound at or below the substrate. The step of detecting the movement of the test compound in the tissue model may comprise detecting the test compound in the purified biocompatible mucus layer, the cell population, and/or in contact with a surface of the substrate.

The test compound may be detected at the luminal surface and/or at the basolateral surface of the tissue model. The presence of the test compound at the basolateral surface indicates the ability of the compound to be absorbed through the tissue explant.

Detecting the test compound may be performed by any suitable means, for example physical, chemical or visual means. Examples include, without limitation, antibody assays, mass spectrometry, colorimetric assays, liquid chromatography, nucleic acid sequencing, SDS PAGE, Western blotting, in situ hybridisation. A suitable method may be selected by the person skilled in the art based upon the nature of the test compound.

The tissue model may be a stomach tissue model, small intestine tissue model, large intestine tissue model, gastro-intestinal tissue model, female reproductive tissue model, nasal tissue model, optic tissue model and/or airway tissue model as described herein.

In an embodiment, the method for determining or predicting absorption of a test compound through a tissue model may be used to determine or predict the effect of food on absorption of a compound into gastro-intestinal tissue. In such a method, the tissue model may be a stomach tissue model, a small intestine tissue model, a large intestine tissue model or a gastro-intestinal tissue model. The method may comprise the step of applying food to the purified biocompatible mucus or fluid layer of the model. The food may be digested or non-digested food. The step of contacting the tissue model with food and a test compound may be separate, or may be simultaneous. Where separate, the test compound may be contacted with the tissue model before or after contact of the tissue model with food. The method may comprise testing the difference in absorption of a compound of interest in the presence or absence of food.

In an embodiment, the method for determining or predicting absorption of a test compound through a tissue model may be used to determine or predict the effect of native intestinal media on absorption of a compound into gastro-intestinal tissue. In such a method, the tissue model may be a stomach tissue model, a small intestine tissue model, a large intestine tissue model or a gastro-intestinal tissue model. The method may comprise the step of applying native intestinal media to the purified biocompatible mucus or fluid layer of the model. The step of contacting the tissue model with native intestinal media and a test compound may be separate, or may be simultaneous. Where separate, the test compound may be contacted with the tissue model before or after contact of the tissue model with native intestinal media. The method may comprise testing the difference in absorption of a compound of interest in the presence or absence of native intestinal media.

The absorption of a test compound into a tissue model may be tested at different time points. This will provide information regarding the rate of absorption of a test compound by the tissue model, including the rate of absorption through different layers of a tissue model. Such a method can be used to predict the rate of absorption through native tissue.

The method of determining or predicting absorption of a test compound as described herein may be used to determine or predict the perfusion rate of the test compound in the tissue explant. The presence of the test compound at the basolateral surface indicates ability of the compound to be perfused in the tissue model. By detecting the presence of the test compound at different time points, a rate of perfusion may be obtained.

The invention may also provide a method for determining the effect of a test compound on a tissue model, wherein the tissue model comprises i) a substrate comprising a cell support portion; ii) a cell population in contact with the cell support portion of the substrate; and iii) purified biocompatible mucus, in contact with the cell; and wherein the method comprises contacting the purified biocompatible mucus with the test compound, and detecting an effect of the test compound on the tissue model. The method may comprise contacting the tissue model with a test compound, and contacting the tissue model with a control substance, to compare an effect of the test compound on the tissue model. The method can be used to predict an effect of the test compound on native tissue.

The tissue model used in a method of determining the effect of a test compound may comprise purified biocompatible mucus, a composition comprising purified biocompatible mucus as described in the first aspect of the invention. The tissue model may be as described in the second aspect of the invention.

Determining the effect of a the test compound may comprise determining the effect of the test compound on local tissue toxicity, genetic modification of tissue, temporary change of tissue permeability, modulation of mucus or microbiome, and modulation of hormone production and/or secretion, general health of tissue cells, viability of all or part of the tissue, for example the condition of the purified mucin layer (e.g in terms of cell viability, cytokine production, or trans epithelial electrical resistance). The condition of the cell population may be assessed in terms of determining the number of live or dead cells, or factor such as presence or absence of cellular markers or cell membrane permeability. Assays include any cell viability assay available to the skilled person for example flow cytometry, high content imaging, colorimetric tetrazolium reagents, resazurin reduction and protease substrates generating a fluorescent signal, the luminogenic ATP assay, fluorescence microscopy, real-time assay to monitor live cells for days in culture, a cell titer blue assay.

The tissue model used in a method of determining the effect of a test compound may be a stomach tissue model, small intestine tissue model, large intestine tissue model, gastro-intestinal tissue model, female reproductive tissue model, nasal tissue model, optic tissue model and/or airway tissue model as described herein.

The method of determining the effect of a test compound may be used to determine or predict the effect of food on the effect of the compound. In such a method, the tissue model may be a stomach tissue model, a small intestine tissue model, a large intestine tissue model or a gastro-intestinal tissue model. The method may comprise the step of applying food to the mucin or fluid layer of the model. The food may be digested or non-digested food. The step of contacting the tissue model with food and a test compound may be separate, or may be simultaneous. Where separate, the test compound may be contacted with the tissue model before or after contact of the tissue model with food. The method may comprise testing the difference in absorption of a compound of interest in the presence or absence of food.

In an embodiment, the method of determining the effect of a test compound may be used to determine or predict the effect of native intestinal media on the effect of the compound. In such a method, the tissue model may be a stomach tissue model, a small intestine tissue model, a large intestine tissue model or a gastro-intestinal tissue model. The method may comprise the step of applying native gastrointestinal media to the mucin or fluid layer of the model. The step of contacting the tissue model with native intestinal media and a test compound may be separate, or may be simultaneous. Where separate, the test compound may be contacted with the tissue model before or after contact of the tissue model with native intestinal media. The method may comprise testing the difference in absorption of a compound of interest in the presence or absence of native intestinal media.

The effect of a test compound into a tissue model may be tested at different time points. This will provide information regarding the rate of effect of a test compound on the tissue model, including the rate of effect on different layers of a tissue model.

The above methods may be useful in identifying optimum or suitable drug delivery mechanisms, drug formulations, and drug effects on the body. The above methods may also be suitable for identifying modulators of protein, lipid, or fat digestion for use in the food/nutraceuticals industry. The above methods may also be useful in testing the effect of factors such as food, mealtimes, etc on drug efficacy.

A kit may be provided for use in a method of the present invention. A kit may comprise, in a suitable container, a tissue model as described herein, and optionally a substrate, a buffer, reagents, instructions for use, and other standard ingredients well known in the art.

A test compound for use in the present invention may be any compound which has or may have a biological activity, including for example but not limited to small molecules, organic molecules, antibodies, peptides, proteins, hormones, antagonists, and nucleic acids (e.g. antisense, siRNA, shRNA, RNAi, expressible coding sequences), saccharides, fatty acids, steroids, purines, pyrimidines, derivatives, structural analogs, bacteria and fungi, or extracts thereof, plant and animal extracts. A test compound may be a pharmaceutical agent. A test compound may include a combination or two or more compounds.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLES

### Production of Reconstituted Mucus

Mucus scrapings from the porcine small intestine were mixed with an enzyme inhibitor cocktail (Table 5) at a 3:1, inhibitor buffer to mucus, ratio to prevent mucin lysis on the behalf of enzymes and other biological components such as bacteria present in the intestinal scrapings. The mucus/inhibitor buffer concoction was homogenised to solubilise the mucin. Homogenisation may depend on the source of the mucus, and may be performed for 0 to 30 minutes. Homogenised material was centrifuged at 10,000 RPM, at 4° C. for 1 hr in 85 ml centrifuge tubes (Eppendorf F-34-6-38 fixed-angle rotor and the Eppendorf Centrifuge 5810 R). The resulting supernatant from this centrifugation was filtered through glass wool, with the pellet being discarded. The supernatant was adjusted to a density of 1.42 g/l using caesium chloride and ultracentrifuged using a P50AT2-941 rotor in a Himac CP100NX ultracentrifuge. Ultracentrifuge tube assembly kit, compatible with the above rotor and centrifuge, was used, this included; 40PA 40 mL, high speed, round bottom tube and S-40AL Caps & Ti rings. Ultracentrifuge, rotor, and tube assemblies were supplied by Hitachi, Ltd. (Hitachi High-Technologies, Maidenhead, UK). This step separates the solubilised mucin from nucleic acids, lipids and proteins via this equilibrium density centrifugation which causes the solution to form a density gradient, which is separated into 8 separate fractions. The heaviest being 8 at the bottom of the tube, and the lightest, fraction 1, being at the top. These fractions have been previously identified via weight. The fractionated sample were pipetted off in series, from 1 to 8, each fraction is around 4 ml.

Each fraction was dialysed using a 19 mm inflated, 12-14 kDa molecular-weight-cut-off (MWCO) dialysis membrane (Medicell Membranes Ltd, London, UK), to remove unwanted chemicals such as components of the inhibitor buffer and caesium chloride. Dialysis was done in a 20 L stirred container filled with deionised (DI) water, with six routine changes of the Di water over 2 days. This step was maintained at 4° C. through use of a cold room.

Following complete dialysis, OD260/280 measurements were taken using an Infinite M200 Pro spectrophotometer (TECAN UK, Reading, UK) to determine the DNA/protein content of the fractions. To determine glycoprotein content, a PAS assay was done using porcine gastric mucin as a standard.

Fractions with the highest glycoprotein content and lowest amounts of DNA/protein were deemed to be the fractions with the mucin, as mucins are a glycoprotein rich in carbohydrate, and were then freeze dried and stored at −20° C. until required. When required 1×PBS (Merck) was added to rehydrate the mucin to the required concentration and, left on a tube roller at 4° C. overnight to hydrate.

The product may be sterilised by the addition of anti-microbial agents during the dialysis process, or through solvent sterilisation of the purified mucus. The sterilisation process may be performed after lyophilization. For solvent sterilisation, solvents such as ethanol, methanol, and IPA may be added to the mucus, mixed and evaporated off in sterile conditions.

### Small Intestinal Absorption Plate Model

Permeation experiments throughout the project were done using dual-chamber microplates consisting of either 96, or 24 chambers. Each chamber is referred to as either basolateral or apical-corresponding to the lower well(s) present in the microplate base, or present as a smaller permeable insert chamber which sits within the base well. The apical chamber inserts from each of these microplates were lined at the bottom with a 3 μM pore size polycarbonate membrane, which separates the upper apical from the basolateral chamber.

In these experiments, the basolateral chamber represents what would be available for uptake, once exiting the basolateral side of the epithelial cells, for villi associated circulatory blood and lymph vessels. The apical chamber represents the lumen of the small intestine.

In all plates simulated digestive fluids (e.g. EPDF) are applied apically containing the substance of interest. In the basolateral chamber is PBS (or any suitable alternative), which represents bodily fluids at the correct pH and osmolarity.

In all plates the basolateral chamber is filled prior to application of fluids to the apical chamber, and the time course ensues immediately following apical fluid application. 100 μL samples are taken from the basolateral chamber at 5, 60, 120 180 and 240 minutes, being replaced with 37° C. pre-warmed PBS (or any suitable alternative). All plates are incubated at 37° C.

In the basolateral chambers of each plate, there are the following volumes of PBS: 300 μL (96 well plate), 600 μL (24 well plate). In the apical chambers of each plate there are the following volumes of simulated digestive fluids and component of interest: 100 μL (96 well plate), 200 μL (24 well plate).

### Cell Monolayer

Caco-2 cells passage 22-36 are grown on the permeable insert membrane (5000 cells per well initially for 96 well plate, 20,000 per well for 24 well plates. After 21 days cells show normal epithelial resistance and transport activity

### Preparation and Application of Reconstituted Mucus in Permeability Validation Against Native Mucus

All permeation validation experiments have been done in 96 well plates. Purified mucus as prepared above was reconstituted in 1×PBS at concentrations of 25, 50 and 75 mg/ml prior to days of experiments and left to hydrate on tube rollers at 4° C. Reconstituted mucus was applied to the apical insert membrane in volumes of; 20, 50 and 70 μL. These correspond to 1.4 mm, 3.5 mm and 4.9 mm respectively.

Drugs were made up at 100UM in simulated digestive fluids and applied (gently) to the mucus layer—thus beginning the time course. Permeations of drugs were done alongside permeations through native porcine small intestinal mucus at volumes of 10, 15 and 20 μL respectively.

### Permeation Validation Data

Correlation analysis of 10 drugs revealed that the best correlations were achieved with 50 μL of 25, 50 and 75 mg/ml, and 70 μL of 50 mg/ml respectively. These concentrations and volumes were used in further analysis with others excluded.

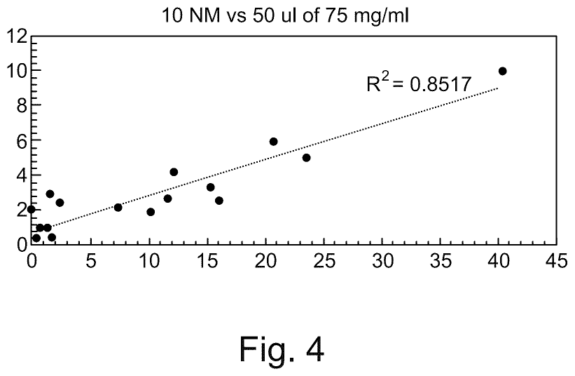

Correlation analysis of 11 drugs revealed positive correlations between the variables, with the strongest being 50 μL of 50 mg/ml, and 50 μL of 75 mg/ml against the three volumes of native mucus (Spearmans Rho R squared, Table 3; FIG. 4 (50 μL of 75 mg/ml). See FIGS. 4, 5 and 6.

### Rheology Validation of Reconstituted Mucus

The purified mucus, as described previously, was reconstituted in phosphate buffered saline (PBS) at a range of concentrations (5-200 mg/ml) overnight at 4° C. Using a Kinexus Pro Rheometer (Malvern, UK) the rheological properties of the rehydrated mucin (reconstituted mucus) was assessed. The properties of native (uncleaned mucus) collected from the small intestine of freshly slaughtered pigs and swiftly frozen, was also assessed once deforested, alongside native mucus that had been through a gentle cleaning process to remove any food debris. The linear-viscoelastic region (LVER) was calculated using an amplitude sweep (0.01 to 600% shear strain) and set at a frequency of 1 Hz. The breakdown (yield point) shear strain (%) were also calculated along with the phase angle, storage modulus (G′), and loss modulus (G″) within the LVER. One-way ANOVA with Tukey's multiple comparisons were used to assess for any statistically significant changes.

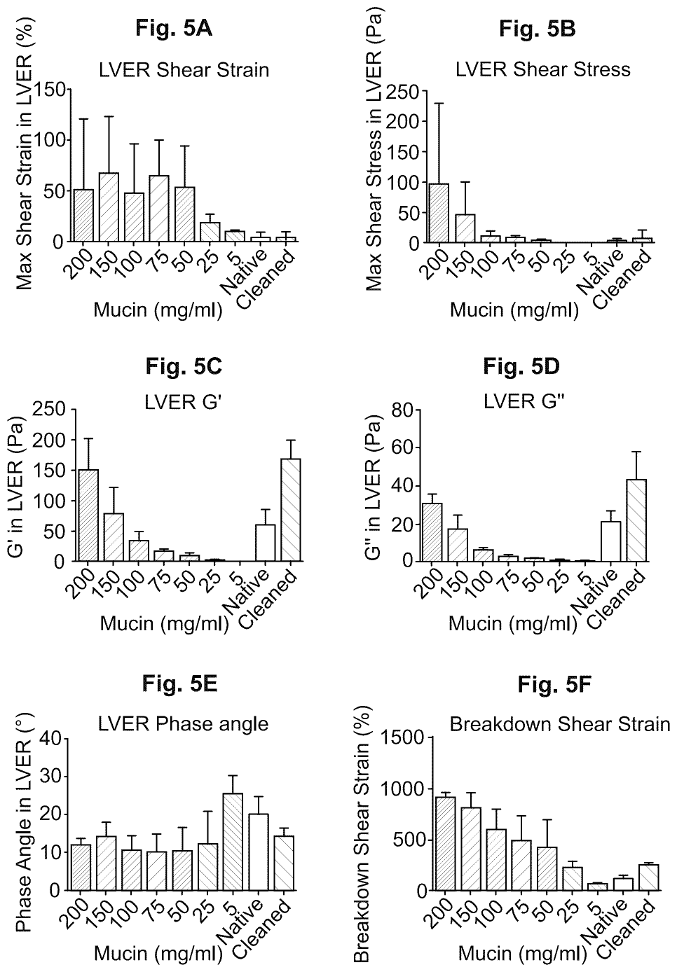

Shear strain and stress in the LVER as well as the yield point were similar in the native and cleaned mucus. There was an increase in the storage and loss modulus for the cleaned mucus compared to the native but the phase angle remained similar. There were no statistical differences between native or cleaned mucus at any of the concentrations of mucin for LVER shear stress, shear strain and phase angle. There were no statistical differences between native mucus and concentrations of mucin below 100 mg/ml for G′ in the LVER, or breakdown shear stress. The only significant differences between the mucin concentrations and native mucus were seen with G″, where only concentration at 100 mg/ml or above did not significantly lower G″ than native (FIG. 5—Rheology Data). FIG. 5 shows the rheological properties of rehydrated mucus compared to native and cleaned porcine small intestinal mucus. Panel A and B show the maximum shear stain and stress respectively in the Linear viscoelastic region. Panel C, D and E show the storage modulus, loss modulus and phase angle within the LVER. Panel F shows the shear strain required to breakdown the down the gel to a viscous liquid (yield point) and allow it to flow. Experiments were repeated a minimum of three times with separate batches of mucus and mucin.

Using breakdown shear strain, LVER, phase angle and G′ as the most relevant aspects for defining rheologically relevant mucus, concentrations at and around 50 mg/ml of mucin are therefore classed as similar.

### Reconstituted Mucus Cell Compatibility

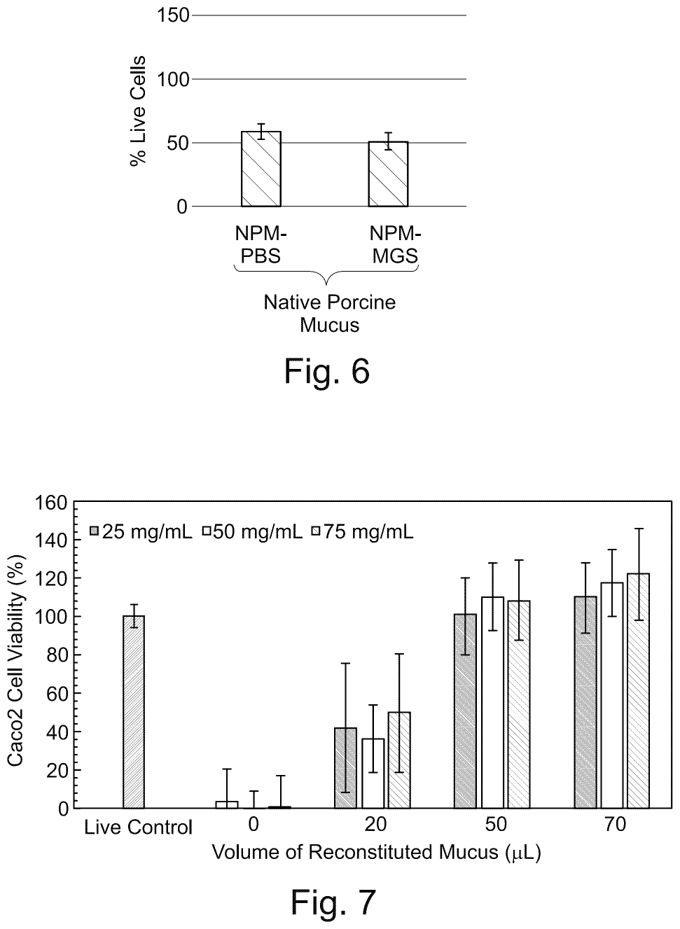

Experiments show that native porcine mucus significantly reduces cell viability in 96 well plate models. (FIG. 6)

In the same model, a range of concentrations and volumes of reconstituted mucus were tested for their compatibility with Caco2 cells with applied small intestinal digestive secretions for a length of 4 hours (FIG. 7).

50 and 70 μL of all concentrations (5-200 mg/ml) were not significantly different from the control, indicating that these protect cells from the simulated digestive secretions in vitro over a time course of 4 hours.

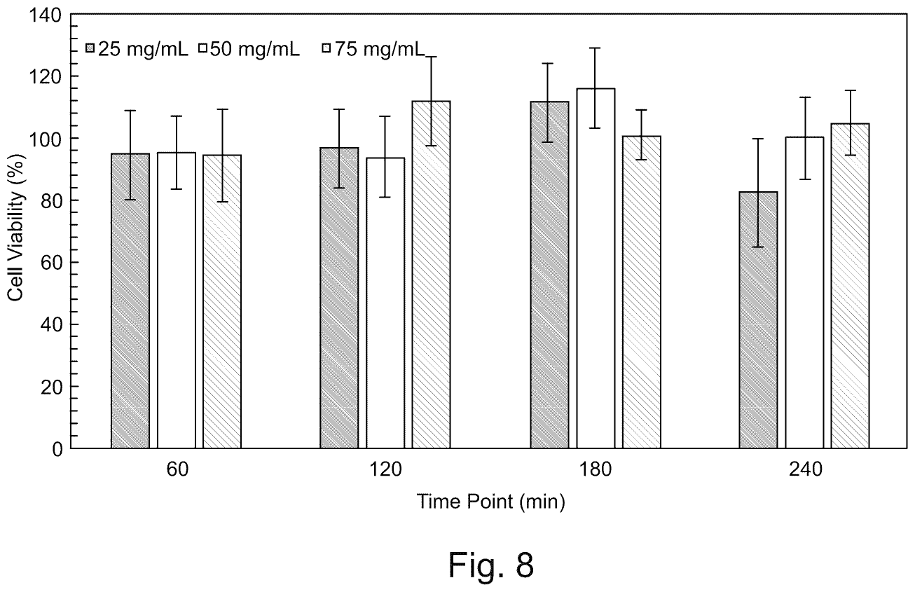

In a 24 well plate model, 50 μL of 25, 50 and 75 mg/ml also protected cells from simulated small intestinal digestive fluids over a 4-hour time course (FIG. 8)

### Small Intestine Model: Summary

Reconstituted mucus is validated rheologically against native mucus.
Reconstituted mucus protects cells from small intestinal digestive fluids for 4 hours in 24 and 96 well plate absorption models.
Reconstituted mucus is validated against porcine native mucus for permeation of pharmaceuticals.

### Gastric Model

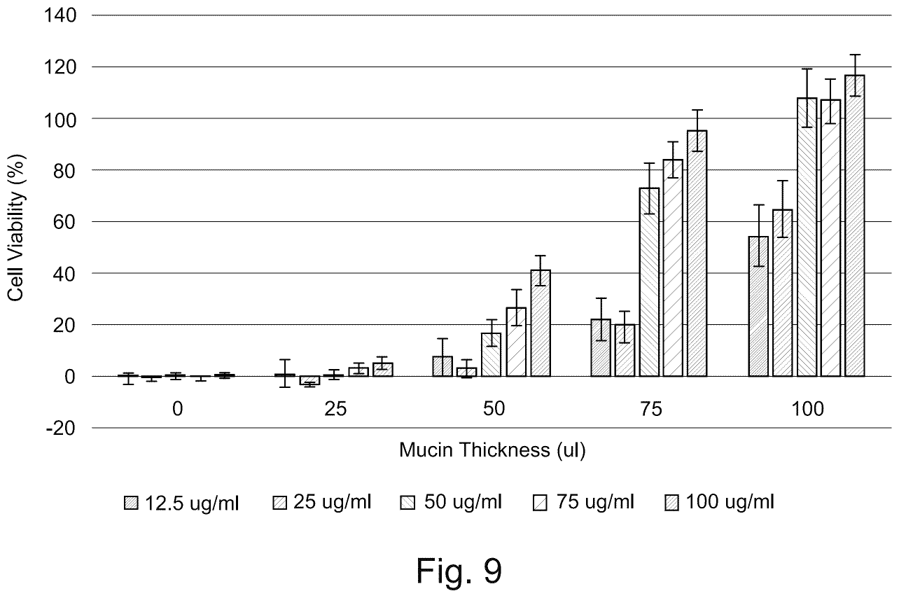

The same system has been used in gastric applications, such as a system for modelling the gastric epithelia using a reconstituted gastric mucin layer with a gastric cell line (CRL-1739) and synthetic gastric secretions. This model has been used to test gastric damage with delivery of non-steroidal anti-inflammatoires. With this model we were able to collect data on cell viability after a two-hour exposure to the test formulations in different conditions. It can be seen that inclusion of a mucus layer protects from cell death, furthermore this allows for gastric secretions to be properly modelled including pepsin, and at pH2.0, and formulations can be identified that are still cytotoxic with the mucus layer. Example data from gastric model is shown in FIG. 9.

### Large Intestinal Model

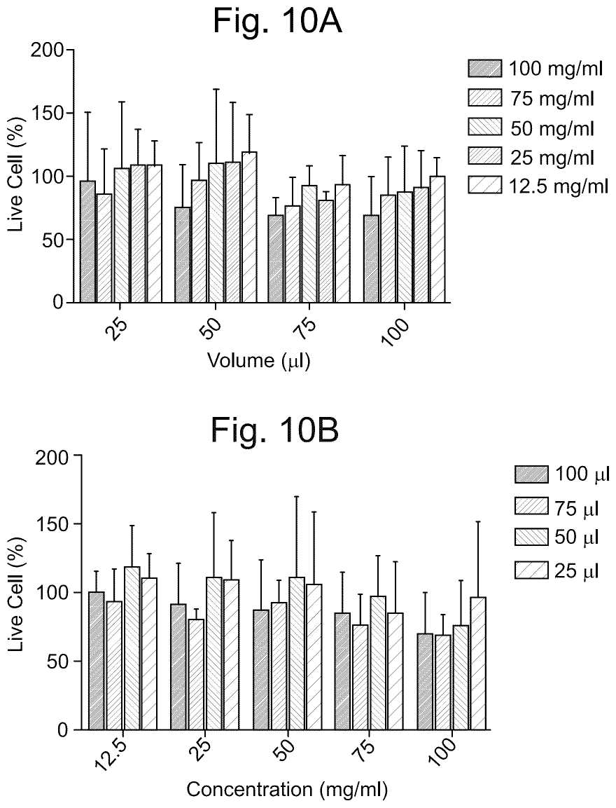

The same method has been applied to a large intestinal model. This system again includes a cell monolayer and a mucus layer in a Transwell system (FIG. 10). However, in order to create an aerobic/anaerobic system, this custom biocompatible 3D printed plate would be placed in a container with a GasPak anaerobic envelope and have an upper section where the apical sides of the Transwells could be accessed, an airtight bottom chamber with O-rings around the Transwells to ensure an airtight fit, a lid to prevent evaporation of media and tubing continuous to the outside of the container to allow air access to the basolateral side of the Transwells to create an aerobic environment. The upper chamber of the transwell would then be inoculated with a bacterial population grown in a 3-stage fermentation model (inoculated with a faecal sample). This is the first system capable of modelling the microbiome, mucus layer and epithelium in a single integrated system.

When transwells containing only a Caco-2 monolayer and media are placed in the above plates in anaerobic conditions (normal anaer & normal anaer 2), there is a slight loss of cell viability compared to cells with media in transwells in normal aerobic conditions (normal aerobic plate).

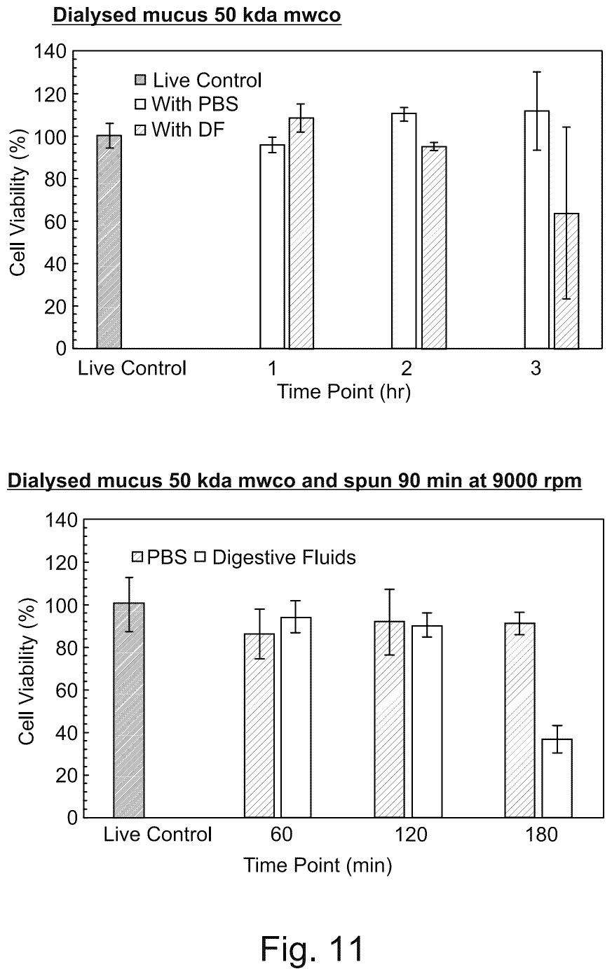

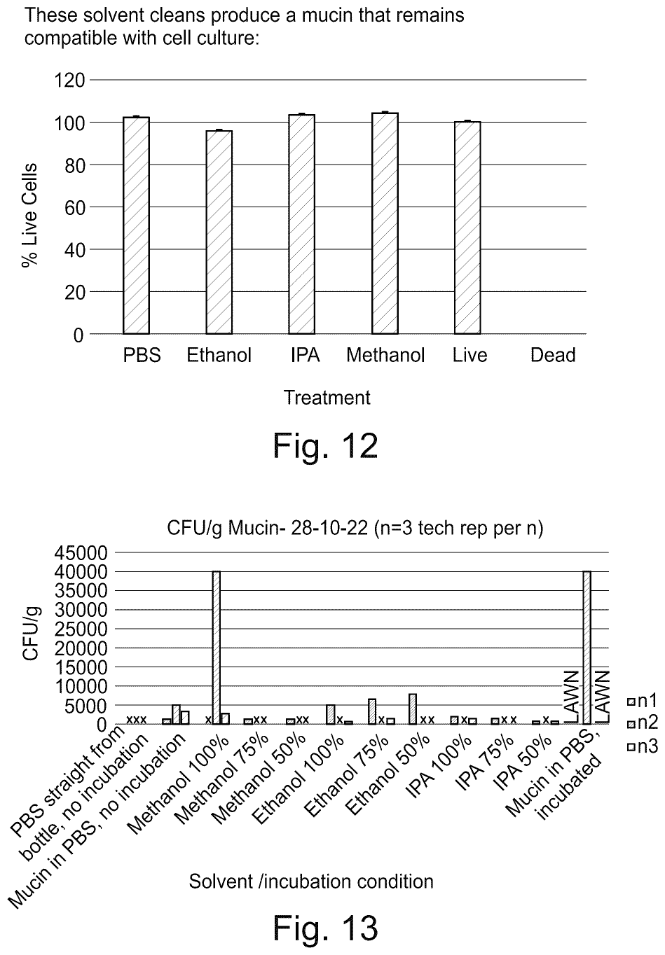

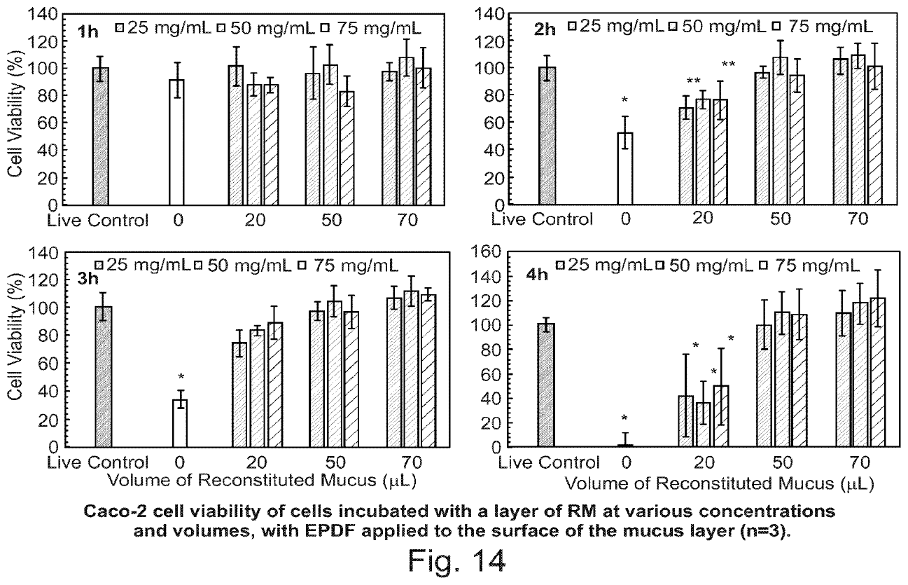

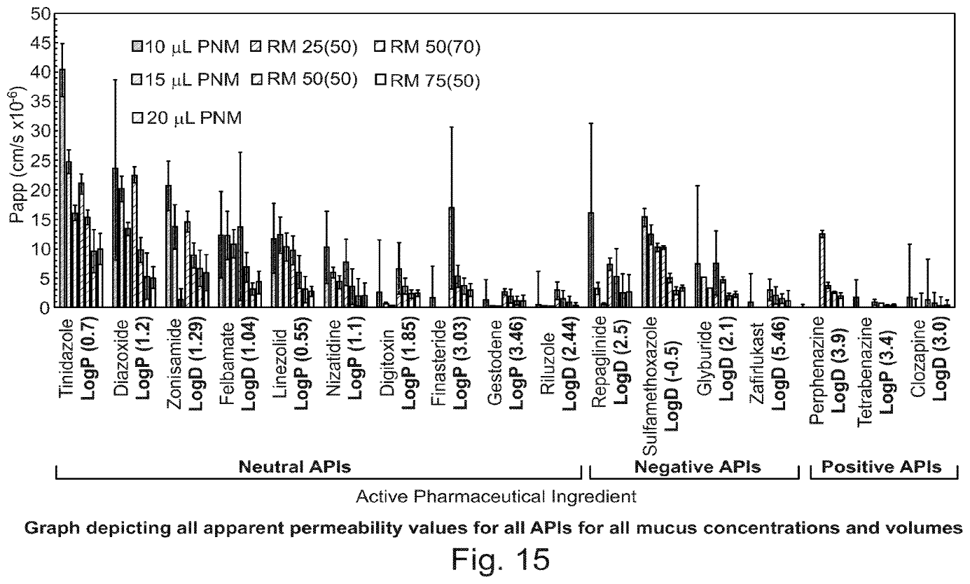

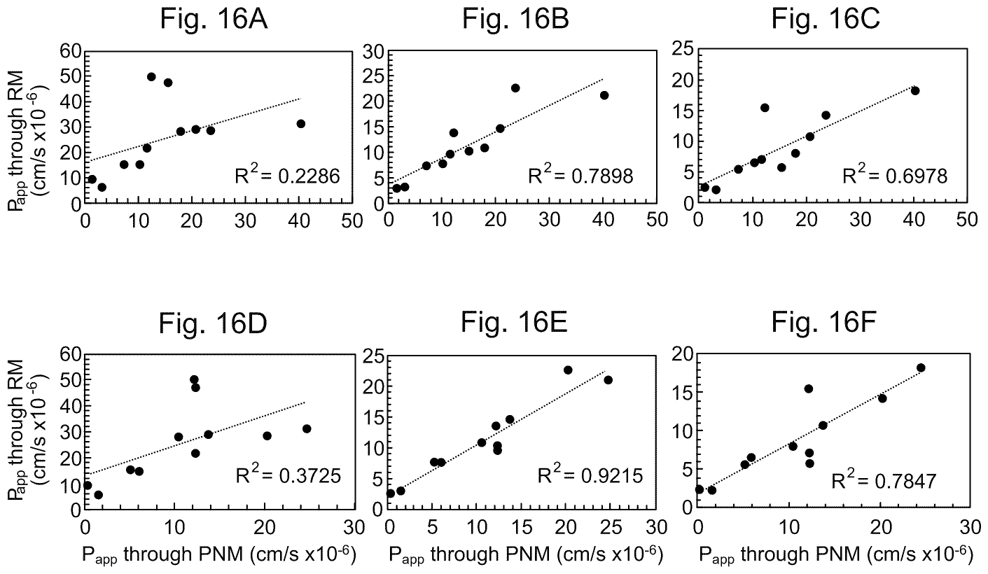

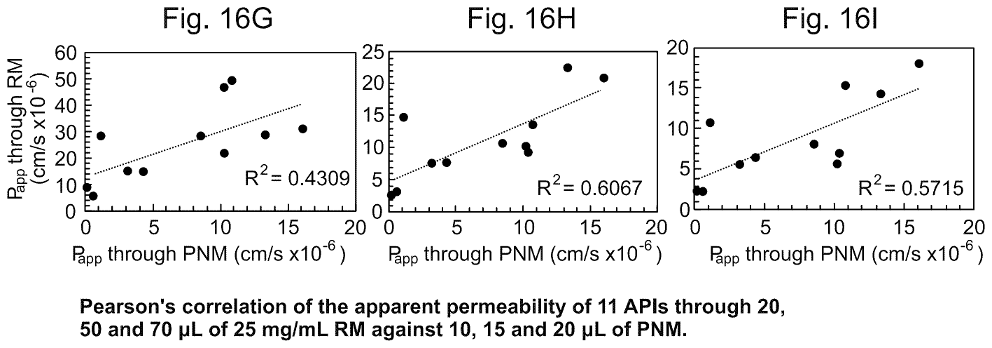

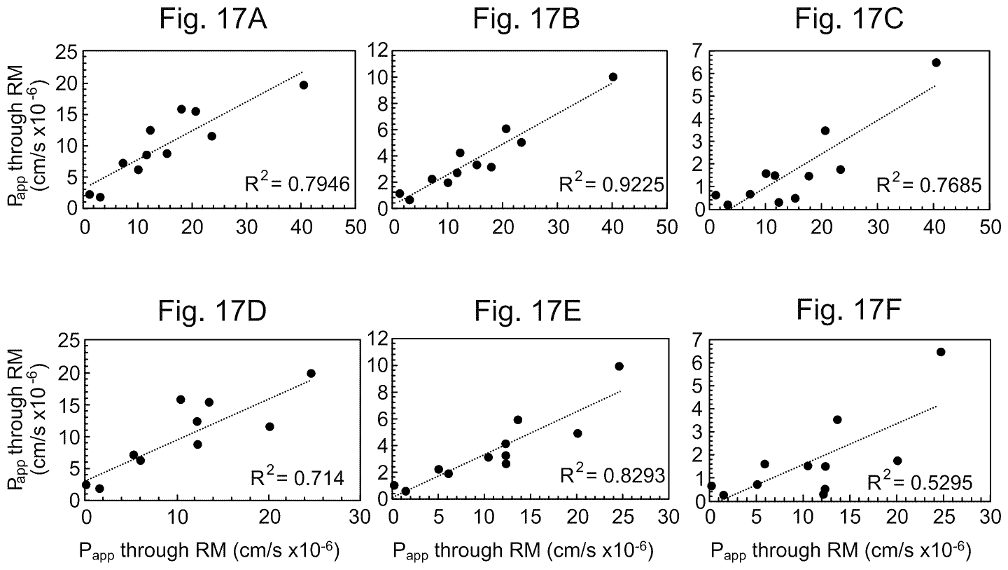

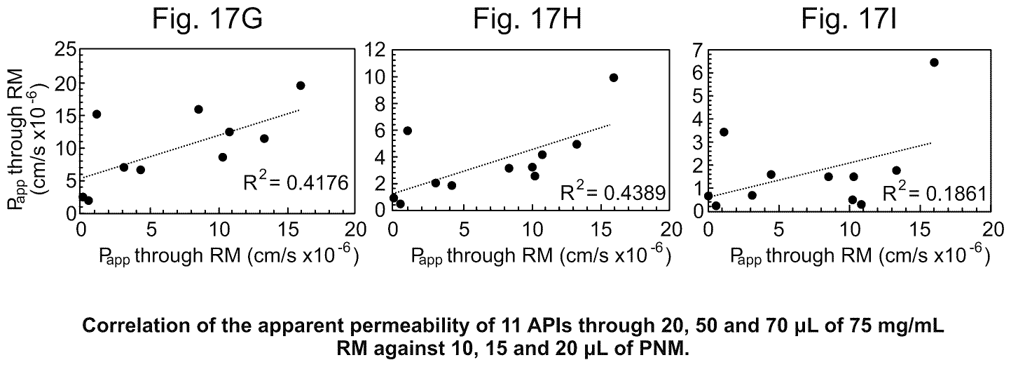

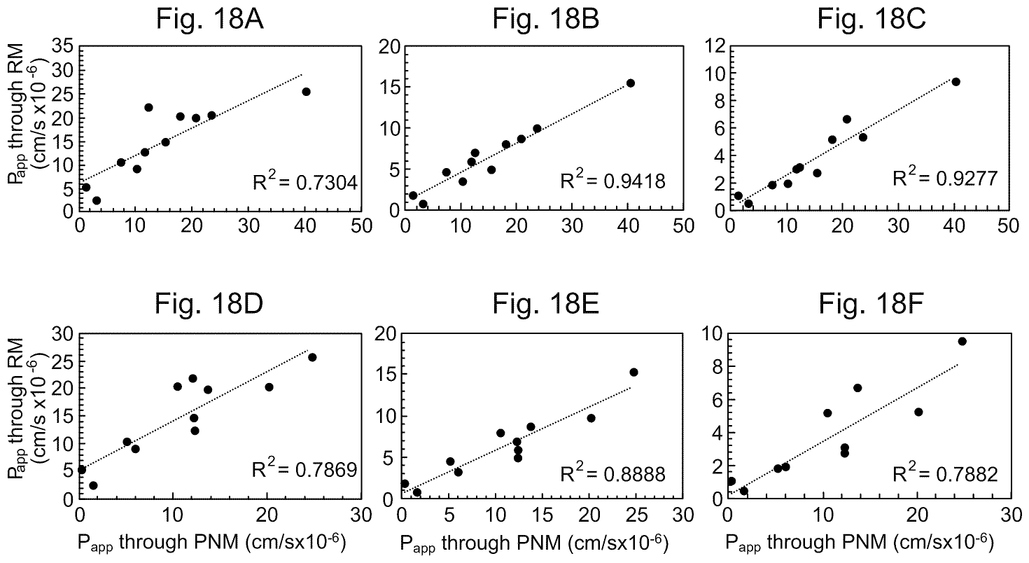

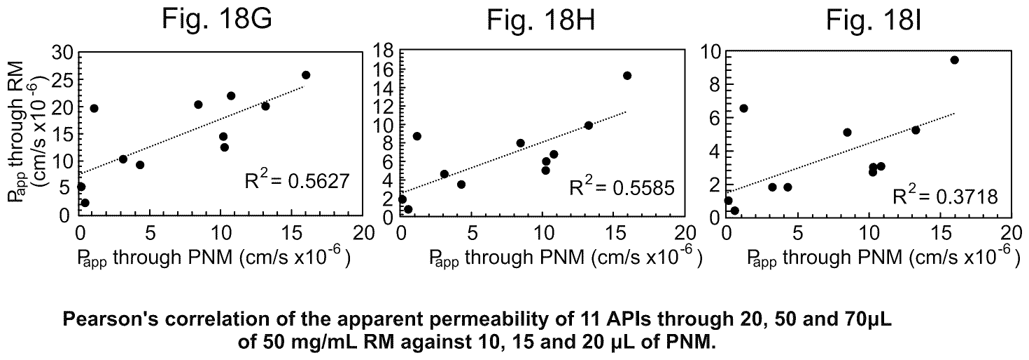

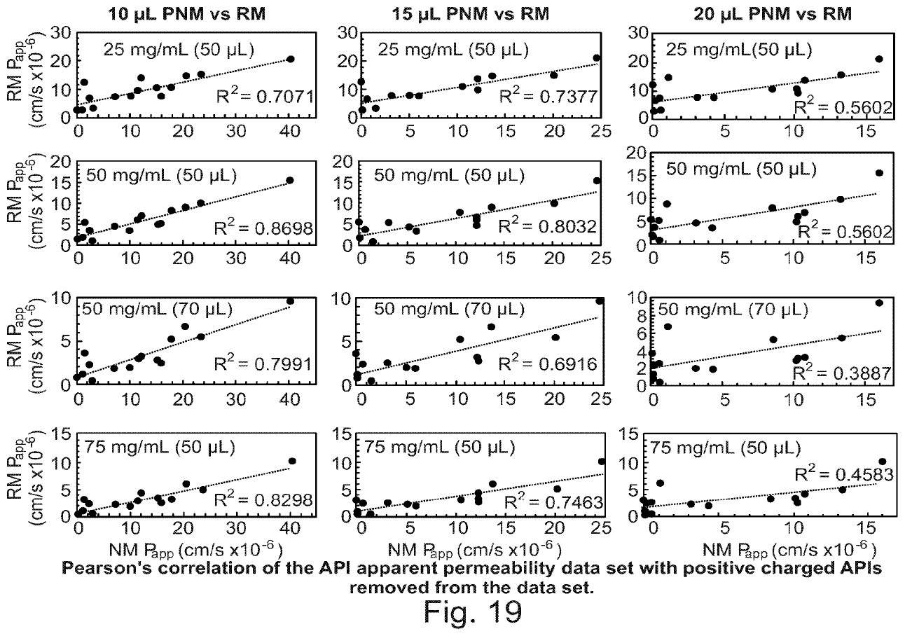

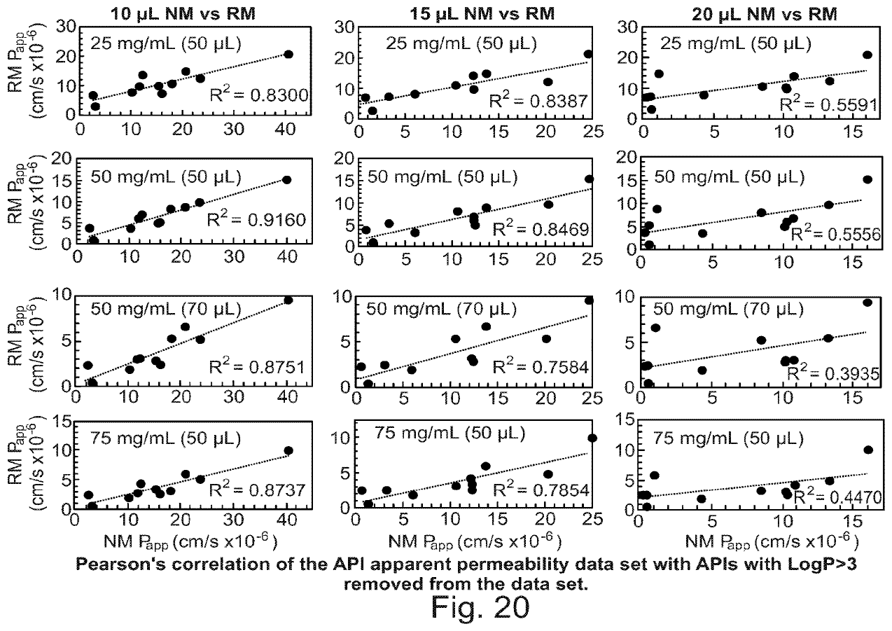

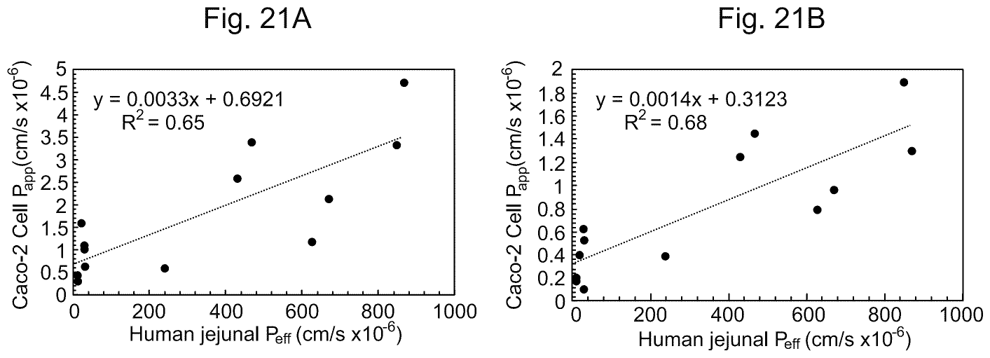

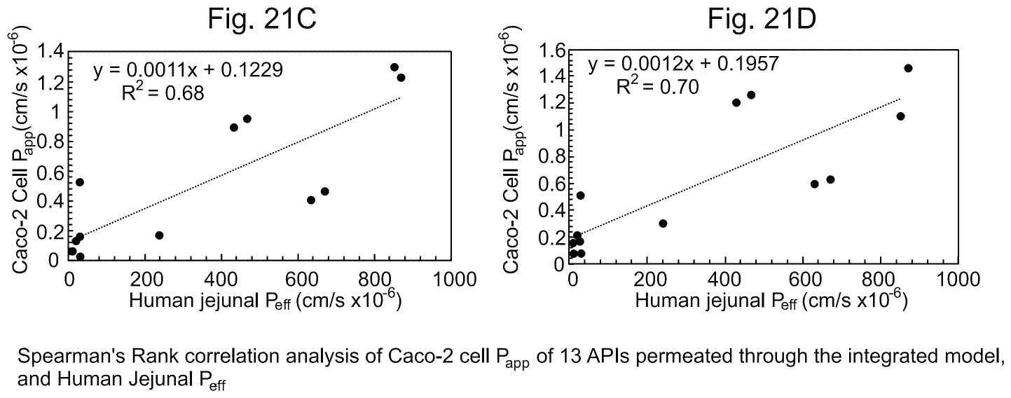

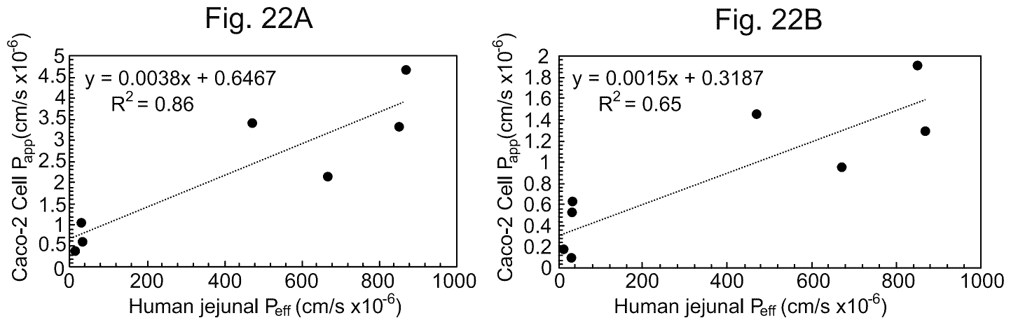

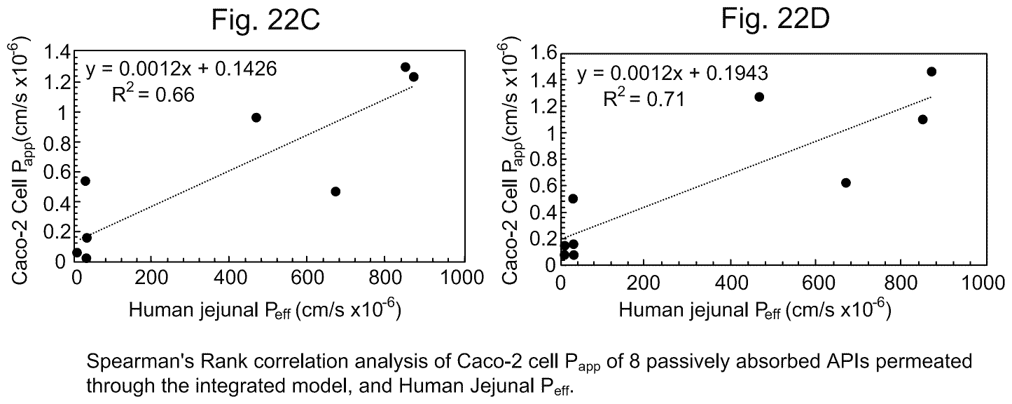

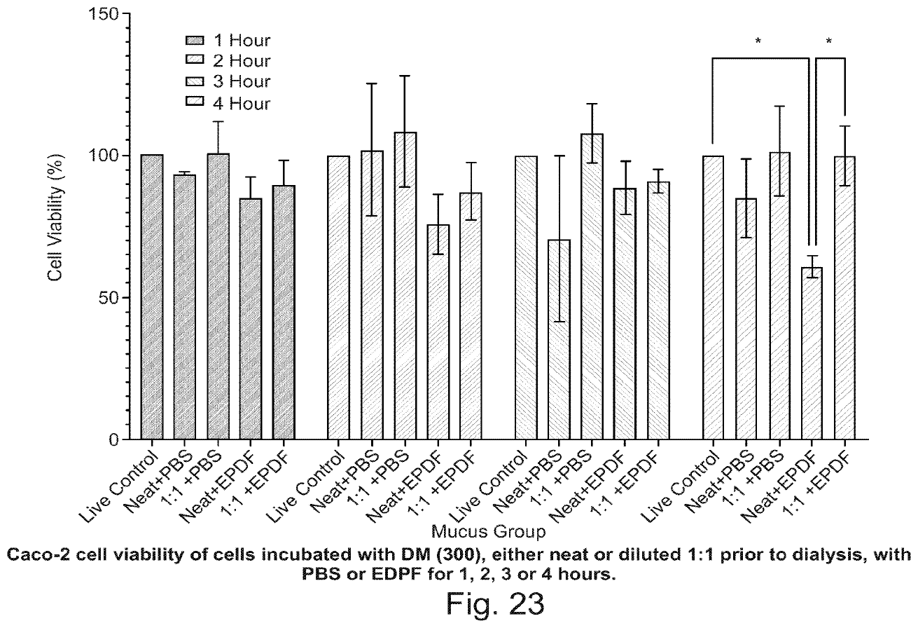

Inclusion of 50 μl of 50 mg/ml reconstituted mucin layer (reconstituted-A) or 50 μl of 100 mg/ml reconstituted mucin layer with 50 μl native porcine mucus layer (native-B) above the cells affected cell viability in aerobic conditions but when the transwells were exposed to anaerobic apical conditions (in the above 3D printed plates, generated by the GasPak) with a reconstituted mucus layer (C) or a native mucus layer (D), the gel was sufficient to protect Caco-2 cells from the anaerobic apical conditions over a 3 hour incubation period (FIG. 11).

Further adding faecal microbial content over the reconstituted (E) or native (F) layers did not negatively impact cell viability.

The fact that there is no significant difference between normal cells in anaerobic conditions, and cells with reconstituted mucus and bacteria shows the viability of the model.

### Airway Model

Calu3 airway epithelial cells were grown in 96 well trans-well cell culture plates. Wells were seeded at 40,000 cells per (100 μl) well and grown until confluent as assessed by eye and trans epithelial electrical resistant measurements and then grown as air liquid interface (ALI) with surface liquid removed and cells fed basolaterally.

Mucus isolated as described above from the stomach of freshly slaughtered pigs containing mainly the mucin gene product of MUC5AC found in the airways was rehydrated in sterile PBS with calcium and magnesium.

Four different volumes of five concentrations of mucin were assessed 25, 50, 75, and 100 μl of either 100, 75, 50, 25, 12.5 mg/ml the mucus applied apically to the cells and incubated for 90 minutes.

Cell viability was assessed via cell titer blue assay. where metabolically active cells reduce resazurin to the dye resorufin, which can be measured spectrophotometrically. Experiments were repeated a minimum of four times (FIG. 10). Significant differences were assessed with a two-way

ANOVA with Tukey's multiple comparison. Panel A shows cell viability groups with volume of liquid added to the apical surface of the cells with concentration of mucin show with the bars. Panel B shows the same data but groups with concentration of mucin applied with the bars showing varying volume.

There were no statistical differences seen between any concentration of the mucin applied at any volume compared none treated cells. Therefore, over the course of 90 minutes the mucin applied did not adversely affect the cell viability.

### Preparation of Purified Mucus

Small intestines of freshly slaughtered pigs were collected from a local abattoir and transported to the laboratory on ice. An incision was made with a scalpel to open up the tissue, and the epithelial tissue surface was gently rinsed with di water. The epithelial surface was gently scraped with a microscope slide to remove mucus. Mucus was added to 50 mDa mwco dialysis tubing neat or diluted down in di water, at a dilution of 1 in 50.

Dialysis tubing was placed in a 10 L bucket of 4 degree DI water and incubated at 4 degrees. Dialysis water was changed 6 times over 2 days

Mucus was collected from the dialysis tubing and centrifuged at 9000 RPM for 90 mins.

Purified mucus was collected and frozen at-20.

### Mucus Permeation Experiments

For permeations of drugs through porcine native mucus (PNM), 0, 10, 15 and 20 μl of PNM was applied to the apical insert membranes of wells in triplicate using a Handy Step repeater pipette (BrandTech Scientific Inc., CT, USA) with a compatible 500 μL Eppendorf Combitips® (Eppendorf, Hamburg, Germany). Nineteen drugs (Table 3) were prepared at 100 UM in simulated small intestinal fluids. The drug solution was added to the apical chamber of test wells at 0 minutes, and 100 μL samples were taken from the basolateral chamber at 5, 60, 120, 180 and 240 minutes at 37° C. Samples were replaced with pre-warmed PBS. For API permeations through reconstituted mucus, the mucus was prepared and made up as described previously, at 12.5, 25, 50, 75 or 100 mg/ml the day before experimentation. Each mucus concentration was added to the apical insert membrane in 0, 20, 50 and 70 μL volumes. Permeation data was analysed via HPLC. All conditions were screened in triplicate.

TABLE 3
List of APIs used in the permeation studies.
API

| | |
|---|---|
| Zonisamide | Finasteride |
| Felbamate | Tetrabenazine |
| Fexofenadine | Perphenazine |
| Diazoxide | Zafirlukast |
| Rivastigmine | Sulfamethoxazole |
| Griseofulvin | Linezolid |
| Repaglinide | Tinidazole |
| Naproxen | Gestodene |
| Clozapine | Glyburide |
| Finasteride | Riluzole |
| Tetrabenazine | Digitoxin |

### Caco-2 Cell Culture

All tissue culture was carried out under sterile conditions using a S@FEFLOW 1.2 Biosafety cabinet (Bio Air Instruments, Pavia, Italy). Caco-2 cells were acquired from ATCC (ATCC® HTB-37™) (American Type Culture Selection, VA, USA). Cells arrived frozen and following manufacturer's instructions were thawed and seeded onto a 25 cm2 Nunc™ EasYFlask™ (Thermo Scientific, NY, USA). Upon reaching around 80% confluence, cells were then further sub-cultured into a 75 cm² flasks Nunc™ EasYFlask™. Medium exchange was carried out every other day, replacing 7 mL and 20 mL respectively for 25 cm² and 75 cm² flasks. Medium used for Caco-2 cell growth was Dulbecco's Modified Eagle Medium—High Glucose (Sigma Aldrich; D5797-500ML), supplemented with 50 mL Foetal Calf Serum, 5 mL of 200 mM L-Glutamine, 5 mL of Penicillin/Streptomycin (Penicillin: 5 mg/ml; Streptomycin: 10 mg/mL) and 5 mL of Modified Eagle Medium Non-essential Amino Acid Solution (100×) (Merck; M7145). Cultures were incubated in an MCO-20AIC CO² incubator (SANYO Electric Co., Ltd, Japan) at 37° C. at 5% CO². Passage number ranged from 22-38.

For 96 well plate experiments, Caco-2 cells were grown on the apical surface of the 3 μm pore size polycarbonate membrane of 96 dual-chamber microplates (HTS Transwell®—96 Well Permeable Supports With barcode, Lid, Receiver Plate; Corning Inc., ME, USA) for a minimum of 21 days. The Caco-2 cells were sub-cultured from 75 cm2 flasks according to ATCC and seeded at 80,000 cells per cm², with the volume of media in each apical well at 100 μL. 300 μL of media was added to the basolateral chambers.

### Integrated Model Experiments

Prior to the day of the experiment, RM was prepared as previously at 25, 50 and 75 mg/mL. On the day of the experiment, the Caco-2 cells, grown for a minimum of 21 days, were moved to a clean, sterile receiver plate by transferring over the apical scaffold. Growth medium was then removed from the apical chambers with a multichannel pipette. 300 μL 1×PBS was added to the basolateral chamber of all test wells. All outer wells were excluded from testing due to edge effects. To these wells, 300 μL of 1×PBS was added to the basolateral chamber, and 100 μL to the apical chamber, to reduce edge effects within the experiment. For test wells, four different RM conditions were used in analysis. These were 25 (50), 50 (50), 50 (70) and 75 (50), where X (X) is equivalent to X mg/mL (X μL). Individual concentrations and volumes were added to the Caco-2 monolayers delicately with a repeater pipette, and left for 5 minutes to settle. 16 APIs (Table 4) were made up at 100 UM in EPDF and 100 μL was delicately applied to the mucus layers with a 200 μL multichannel pipette at TO. The plate was then incubated at 37° C. for 4 hours, with 100 μL samples taken from the basolateral chamber at 60, 120, 180, and 240 minutes and replaced with pre-warmed 37° C. 1×PBS. Samples were incubated at 37° C. overnight, re-suspended in 50% methanol and analysed via HPLC using the Ammonium Acetate Method.

At the end of the experiment, the EPDF was removed from the apical chamber, being careful not to disturb the mucus layer. With the EPDF removed, the apical chamber was gently washed with 1×PBS by pipetting to remove the mucus layer. The cells were washed a minimum of two times. The apical scaffold was then transferred into another sterile receiver plate and a CellTitre-Blue assay was done. All conditions were screened in triplicate.

TABLE 4
Drugs used in the Integrated Model permeations
API

| | | |
|---|---|---|
| Diazoxide | Tinidazole | Glyburide |
| Felbamate | Zonisamide | Riluzole |
| Sulfamethoxazole | Gestodene | Nizatidine |
| Linezolid | Finasteride | Clozapine |
| Repaglinide | Tetrabenazine | |
| Perphenazine | Digitoxin | |

TABLE 5
Protease inhibitor cocktail used for reconstituted mucin production.
Inhibition mechanisms are detailed in the central column

| Component | Inhibition Mechanism | Concentration/Amount | |
|---|---|---|---|
| Phenylmethylsulfonyl fluoride | Irreversibly inhibits serine proteases such as | 1 | mM |
| (PMSF) | trypsin and chymotrypsin by adding a | | |
| | covalently linked sulphonyl group to the serine | | |
| | of the active site (368). | | |
| lodoacetamide | Irreversibly inhibits serine proteases such as | 50 | mM |
| | papain by adding a covalently linked | | |
| | carbamidomethyl group to the cysteine of the | | |
| | active site (369) | | |
| α-amino hexanoic acid | Inhibits plasmin, a serine protease important for | 100 | mM |
| | control of fibrinolysis, by blocking lysine binding | | |
| | sites on the plasmin zymogen, plasminogen, | | |
| | preventing its conversion into plasmin(370). | | |
| Benzamidine hydrochloride | Potent competitive inhibitor of trypsin and other | 5 | mM |
| | serine proteases. Two amidines present in the | | |
| | structure of benzamidine HCl mimic side chains | | |
| | of the substrates (371). | | |
| EDTA in 0.5 mM in Tris HCl pH 8 | Inhibits metalloproteases by scavenging metal | 10 | mM |
| | ions (372). | | |
| Soybean Trypsin Inhibitor | Inhibits trypsin by forming a 1:1 stoichiometric | 1 | mg/ml |
| | complex with the active site (373), reaction is | | |
| | reversible (374). | | |

1. A tissue model, comprising in discrete layers:
i. a substrate having a cell support portion;
ii. a cell population provided on the substrate;
iii. a purified, biocompatible mucus;
iv. optionally, a physiological fluid provided on the mucus layer and/or below the substrate layer,
wherein the purified, biocompatible mucus: i) substantially lacks nucleic acids, protein or lipids or molecules below 100 kDa, preferably 70 kDa; and ii) comprises mucin, wherein the mucin has the ability to form a gel.

2. The tissue model according to claim 1, wherein the purified, biocompatible mucus is a gel.

3. The tissue model according to claim 1, wherein the purified, biocompatible mucus does not contain a synthetic polymer and/or is not substantially denatured, hydrolysed or degraded.

4. The tissue model according to claim 1, wherein the purified, biocompatible mucus is provided in combination with one or more of a lipid, salt, protein, nucleic acid (DNA or RNA), carbohydrate, glycoprotein, cell, and water.

5. The tissue model according to claim 1, wherein the cells of the cell population are, or are derived from, ileum, jejunum, stomach, duodenum, oesophagus, buccal, lingual or colon of the gastrointestinal tract, or from the nose, lung, bronchi, bronchioles, or mouth; or from the cervix or vagina, or from the eye; or any combination thereof.

6. The tissue model according to claim 1, wherein the fluid is digestive fluid, saliva, vaginal secretion, tears, or airways secretions, a synthetic fluid, or a microbial cell population; or any combination thereof.

7. The tissue model according to claim 1, wherein the tissue model is a stomach tissue model, a small intestine tissue model, a large intestine tissue model or a gastro-intestinal tissue model.

8. A combination of two or more tissue models according to claim 1, wherein the two or more tissue models are placed in series, and preferably wherein the two or more tissue models are each selected from the group consisting of an airway model, a stomach tissue model, a small intestine tissue model, and a large intestine tissue model.

9. A purified, biocompatible mucus, wherein the mucus: i) substantially lacks nucleic acids, protein or lipids or molecules below 100 kDa, preferably 70 kDa; and ii) comprises mucin, wherein the mucin has the ability to form a gel.

10. The purified, biocompatible mucus according to claim 9, wherein the purified, biocompatible mucus is a gel.

11. The purified, biocompatible mucus according to claim 9 wherein the purified, biocompatible mucus does not contain a synthetic polymer.

12. The purified, biocompatible mucus according to claim 9, wherein the mucin is not substantially denatured, hydrolysed or degraded.

13. The purified, biocompatible mucus according to claim 9, further comprising one or more of: lipid, salts, protein, nucleic acid (DNA or RNA), carbohydrates, glycoproteins, cells, and water.

14. A method for determining or predicting absorption of a test compound through the tissue model of claim 1; wherein the method comprises contacting the purified biocompatible mucus with the test compound, and detecting movement of the test compound in the tissue model.

15. The method according to claim 14, wherein the method comprises detecting and optionally determining the concentration of the test compound in the purified biocompatible mucus layer, the cell population, in contact with a surface of the substrate, below the substrate, at a luminal surface and/or at a basolateral surface.

16. The method according to claim 14, wherein the test compound is detected by a binding assay, mass spectrometry, a colorimetric assay, liquid chromatography, nucleic acid sequencing, SDS PAGE, Western blotting, or in situ hybridisation.

17. The method according to claim 16, wherein the test compound is a small molecule, an organic molecule, antibody, peptide, protein, hormone, antagonist, nucleic acid (e.g. antisense, siRNA, shRNA, RNAi, expressible coding sequences), saccharide, fatty acid, steroid, purine, pyrimidine; derivatives or structural analogs thereof; bacteria, fungi, or extracts thereof; plant or animal extract; or any combination of two or more thereof.

18. The method according to claim 14, wherein the test compound is a pharmaceutical agent.

19. The method according to claim 14, for use in any one or more of: drug absorption testing, drug formulation development, profiling GI damage/side effects, nutrient uptake analysis, drug-microbiome interaction studies, profiling intestinal damage, and pre/pro biotic testing.

20. The method according to claim 14, wherein the method further comprises a step of applying a food and/or intestinal media to the tissue model.

21. The method according to claim 14, wherein the test compound is measured at one or more different time intervals.

22. A kit comprising, in a suitable container, the purified mucus of claim 9, and optionally a substrate, a buffer, a reagent, or instructions for use.

23. A method of purifying mucus, wherein the method comprises:
removal of substantially all nucleic acids, protein and/or lipids, and/or molecules below 100 kDa, preferably below 70 kDa, from the mucus, wherein the step of removal comprises dialysis and/or equilibrium density gradient centrifugation, thereby producing purified mucus.

24. The method according to claim 23, comprising dialysis of the mucus to remove nucleic acids, protein or lipids or molecules under 100 kDa, preferably under 70 kDa; and optionally further comprising separating the mucus.

25. The method according to claim 23, wherein the method further comprises the steps of:
i) providing a sample comprising mucus;
ii) solubilising any mucin in the mucus;
iii) separating from the mucus any nucleic acids, proteins, lipids; and
iv) dialysing the mucus.

26. The method according to claim 25, wherein the separation step comprises equilibrium density gradient centrifugation, and optionally selecting a fraction having a density of 1.3-1.6 g/mL.

27. The method according to claim 23, wherein the method uses a caesium salt density gradient.

28. The method according to claim 23, further comprising providing a tissue sample comprising mucus; and optionally extracting the mucus from the tissue sample.

29. The method according to claim 23, further comprising sterilising one or more components of the purified mucus.

30. The method according to claim 23, further comprising combining the purified mucus with a buffer, a lipid, salt, protein, nucleic acid (DNA or RNA), glycoprotein, cell, and/or water.

31. The method according to claim 23, further comprising placing the purified mucus under conditions to allow gelation of mucin.

32. The method according to claim 23, further comprising lyophilising, storing, and/or combining the purified mucus with a buffer.

33. The method according to claim 23, further comprising applying the purified mucus to a cell population provided on a substrate to form a tissue model, and optionally applying a fluid to the purified mucus.
